(19) United States
(12) Patent Application Publication
(10) Pub. No.: US 2025/0179035 A1
(43) Pub. Date: Jun. 5, 2025

(54) METHOD FOR PREPARING A REACTION PRODUCT, COMPOSITIONS INCLUDING THE REACTION PRODUCT, AND USES OF THE REACTION PRODUCT

(71) Applicant: The Lubrizol Corporation, Wickliffe, OH (US)

(72) Inventors: MARK J McGUINESS, Chagrin Falls, OH (US); KYLE HODGE, Massillon, OH (US); GLENN CORMACK, Willoughby, OH (US); MICHAEL L. CALL, Chardon, OH (US); NICOLAS PROUST, Painesville, OH (US)

(73) Assignee: The Lubrizol Corporation, Wickliffe, OH (US)

(21) Appl. No.: 18/844,483

(22) PCT Filed: Mar. 10, 2023
(86) PCT No.: PCT/US2023/014934

§ 371 Date: Sep. 6, 2024

Related U.S. Application Data
(60) Provisional application No. 63/318,872, filed on Mar. 11, 2022.

(51) Int. Cl. C07D 263/06

(52) U.S. Cl. CPC C07D 263/06

(57) ABSTRACT

Disclosed is a process for preparing a reaction product, the process including adding 1-amino-2-propanol into a liquid form of formaldehyde to form an intermediate reaction mixture, wherein the molar ratio of formaldehyde to 1-amino-2-propanol is from 1.4:1 to less than 2:1, and subsequently subjecting the intermediate reaction mixture to reactive distillation to form the reaction product, wherein the predominant compound present in the reaction product is 3,3′-methylenebis[5-methyloxazolidine]. Compositions prepared according to the process are also disclosed.

The disclosed technology relates to a method of preparing a reaction product including 3,3′-methylenebis[5-methyloxazolidine], compositions including the reaction product, and uses of the reaction product. The reaction product and/or 3,3′-methylenebis[5-methyloxazolidine] may be used as a scavenger for sulfur-containing compounds, such as hydrogen sulfide.

Removal of sulfur-containing compounds, such as hydrogen sulfide, from fluids has presented a challenge in many industries for some time. In this context, the term “removal” and its analogs mean removal, capturing, and/or otherwise rendering the sulfur-containing compound inert and/or incapable of deleteriously impacting a fluid system; for example, but without limitation, the sulfur-containing compound may or may not actually be removed from the fluid, but instead may be at least partially captured/rendered inert, such that the sulfur-containing compound cannot have a significant negative effect on fluid processing equipment. Removal of sulfur-containing compounds from fluids is sometimes known colloquially as “sulfur scavenging”. For example, removing hydrogen sulfide from hydrocarbon streams is an important step in processing hydrocarbon streams in the petroleum industry. Other sulfur-containing compounds which may need to be removed from hydrocarbon streams include organosulfur compounds having a sulfhydryl (—SH) group, such as thiols (R—SH), thiol carboxylic acids (RCO—SH), dithio acids (RCS—SH), and related compounds.

Bisoxazolidines are known sulfur scavengers, but the processes conventionally used to make such compounds can present safety and/or environmental concerns. For example, many conventional processes used to make bisoxazolidines utilize paraformaldehyde (a solid substance usually in powder form) and 1-amino-2-propanol as reactants. In these processes, the paraformaldehyde is added to the 1-amino-2-propanol, which must be done in multiple additions in order to control the heat of reaction, and the reaction and subsequent stripping may take up to about a day to reach completion.

Handling paraformaldehyde in powder form may present safety concerns for workers, and may also present environmental concerns, which may be mitigated using process controls and worker training. The heat produced by the reaction of paraformaldehyde and 1-amino-2-propanol may also present challenges and concerns which may require additional mitigation strategies.

The disclosed technology at least partially mitigates these concerns by providing an alternative reaction scheme to produce 3,3′-methylenebis[5-methyloxazolidine], as discussed in detail below.

The subject matter disclosed herein provides a process for preparing a reaction product, the process comprising adding 1-amino-2-propanol into a liquid form of formaldehyde to form an intermediate reaction mixture, wherein the molar ratio of formaldehyde to 1-amino-2-propanol is from 1.4:1 to less than 2:1, and subsequently subjecting the intermediate reaction mixture to reactive distillation to form the reaction product, wherein the predominant compound present in the reaction product is 3,3′-methylenebis[5-methyloxazolidine].

Also provided is a composition comprising a reaction product, wherein the reaction product is prepared by adding 1-amino-2-propanol into a liquid form of formaldehyde to form an intermediate reaction mixture, wherein the molar ratio of formaldehyde to 1-amino-2-propanol is from 1.4:1 to less than 2:1, and subsequently subjecting the intermediate reaction mixture to reactive distillation to form the reaction product, wherein the predominant compound present in the reaction product is 3,3′-methylenebis[5-methyloxazolidine].

Using a liquid form of formaldehyde, as opposed to the conventional paraformaldehyde, may result in a decrease in reaction time, an increase in product output per unit of time, and/or the possibility of running a continuous reaction process. Furthermore, the liquid form of formaldehyde is significantly easier to handle than the solid/powder form of paraformaldehyde, reducing safety and/or environmental concerns.

Adding the 1-amino-2-propanol into the liquid form of formaldehyde (as opposed to the conventional process of adding solid/powder paraformaldehyde to the 1-amino-2-propanol) may result in additional benefits, such as isolation of a higher-purity product (i.e., higher concentration of 3,3′-methylenebis[5-methyloxazolidine] in the reaction product) and/or a more controllable heat of reaction. Without wishing to be limited by theory, it is believed that a higher-purity product may result in a more efficacious sulfur scavenger, as the product may deliver more moles of formaldehyde per mole of reaction product, which may in turn result in a net treat rate reduction in order to deliver equivalent sulfur scavenging performance as compared with a lower-purity reaction product, or perhaps an increased efficacy of sulfur scavenging performance at a similar treat rate as compared with a lower-purity reaction product.

### The Following Embodiments of the Present Subject Matter are Contemplated

1. A process for preparing a reaction product, the process comprising adding 1-amino-2-propanol into a liquid form of formaldehyde to form an intermediate reaction mixture, wherein the molar ratio of formaldehyde to 1-amino-2-propanol is from 1.4:1 to less than 2:1, and subsequently subjecting the intermediate reaction mixture to reactive distillation to form the reaction product, wherein the predominant compound present in the reaction product is 3,3′-methylenebis[5-methyloxazolidine].

2. The process of embodiment 1, wherein formation of the intermediate reaction mixture is substantially complete instantaneously after adding the 1-amino-2-propanol into the liquid form of formaldehyde.

3. The process of either embodiment 1 or embodiment 2, wherein the reaction product comprises less than 30 percent by weight of 1,3,5-tris(2-hydroxypropyl) hexahydro-s-triazine, based on the total weight of the reaction product.

4. The process of any one of embodiments 1 to 3, wherein the reaction product has a water content of 10 percent by weight or less, based on the total weight of the reaction product.

5. A composition prepared according to the process of any one of embodiments 1 to 4.

6. A composition comprising a reaction product, wherein the reaction product is prepared by adding 1-amino-2-propanol into a liquid form of formaldehyde to form an intermediate reaction mixture, wherein the molar ratio of formaldehyde to 1-amino-2-propanol is from 1.4:1 to less than 2:1, and subsequently subjecting the intermediate reaction mixture to reactive distillation to form the reaction product, wherein the predominant compound present in the reaction product is 3,3′-methylenebis[5-methyloxazolidine].

7. The composition of embodiment 6, wherein the reaction product comprises less than 30 percent by weight of 1,3,5-tris(2-hydroxypropyl) hexahydro-s-triazine, based on the total weight of the reaction product.

8. The composition of either embodiment 6 or embodiment 7, wherein the reaction product has a water content of 10 percent by weight or less, based on the total weight of the reaction product.

### Various Features and Embodiments of the Present Subject Matter Will be Described Below by Way of Non-Limiting Illustration

As used herein, the indefinite article “a” /“an” is intended to mean one or more than one. As used herein, the phrase “at least one” means one or more than one of the following terms. Thus, “a” /“an” and “at least one” may be used interchangeably. For example “at least one of A, B or C” means that just one of A, B or C may be included, and any mixture of two or more of A, B and C may be included, in alternative embodiments.

As used herein, the transitional term “comprising,” which is synonymous with “including,” “containing,” or “characterized by,” is inclusive or open-ended and does not exclude additional, un-recited elements or method steps. However, in each recitation of “comprising” herein, it is intended that the term also encompass, as alternative embodiments, the phrases “consisting essentially of” and “consisting of,” where “consisting of” excludes any element or step not specified and “consisting essentially of” permits the inclusion of additional un-recited elements or steps that do not materially affect the essential or basic and novel characteristics of the composition or method under consideration.

Provided is a process for preparing a reaction product, the process comprising adding 1-amino-2-propanol into a liquid form of formaldehyde to form an intermediate reaction mixture, wherein the molar ratio of formaldehyde to 1-amino-2-propanol is from 1.4:1 to less than 2:1, and subsequently subjecting the intermediate reaction mixture to reactive distillation to form the reaction product, wherein the predominant compound present in the reaction product is 3,3′-methylenebis[5-methyloxazolidine].

The liquid form of formaldehyde may be any suitable form of formaldehyde provided in liquid form. Non-limiting examples of suitable liquid forms of formaldehyde include formalin (an aqueous form of formaldehyde, such as a 37% solution of formaldehyde in water, perhaps with a minor amount of methanol as a stabilizer) and/or non-aqueous liquid forms of formaldehyde.

In certain embodiments, the molar ratio of formaldehyde to 1-amino-2-propanol is from 1.4:1 to 1.99:1, from 1.4:1 to 1.95:1, from 1.4:1 to 1.9:1, from 1.4:1 to 1.85:1, from 1.4:1 to 1.8:1, from 1.4:1 to 1.75:1, from 1.4:1 to 1.7:1, from 1.4:1 to 1.65:1, from 1.4:1 to 1.6:1, from 1.4:1 to 1.55:1, from 1.4:1 to 1.5:1, from 1.4:1 to 1.45:1, from 1.45:1 to less than 2:1, from 1.45:1 to 1.99:1, from 1.45:1 to 1.95:1, from 1.45:1 to 1.9:1, from 1.45:1 to 1.85:1, from 1.45:1 to 1.8:1, from 1.45:1 to 1.75:1, from 1.45:1 to 1.7:1, from 1.45:1 to 1.65:1, from 1.45:1 to 1.6:1, from 1.45:1 to 1.55:1, from 1.45:1 to 1.5:1, from 1.5:1 to less than 2:1, from 1.5:1 to 1.99:1, from 1.5:1 to 1.95:1, from 1.5:1 to 1.9:1, from 1.5:1 to 1.85:1, from 1.5:1 to 1.8:1, from 1.5:1 to 1.75:1, from 1.5:1 to 1.7:1, from 1.5:1 to 1.65:1, from 1.5:1 to 1.6:1, from 1.5:1 to 1.55:1, from 1.55:1 to less than 2:1, from 1.55:1 to 1.99:1, from 1.55:1 to 1.95:1, from 1.55:1 to 1.9:1, from 1.55:1 to 1.85:1, from 1.55:1 to 1.8:1, from 1.55:1 to 1.75:1, from 1.55:1 to 1.7:1, from 1.55:1 to 1.65:1, from 1.55:1 to 1.6:1, from 1.6:1 to less than 2:1, from 1.6:1 to 1.99:1, from 1.6:1 to 1.95:1, from 1.6:1 to 1.9:1, from 1.6:1 to 1.85:1, from 1.6:1 to 1.8:1, from 1.6:1 to 1.75:1, from 1.6:1 to 1.7:1, from 1.6:1 to 1.65:1, from 1.65:1 to less than 2:1, from 1.65:1 to 1.99:1, from 1.65:1 to 1.95:1, from 1.65:1 to 1.9:1, from 1.65:1 to 1.85:1, from 1.65:1 to 1.8:1, from 1.65:1 to 1.75:1, from 1.65:1 to 1.7:1, from 1.7:1 to less than 2:1, from 1.7:1 to 1.99:1, from 1.7:1 to 1.95:1, from 1.7:1 to 1.9:1, from 1.7:1 to 1.85:1, from 1.7:1 to 1.8:1, from 1.7:1 to 1.75:1, from 1.75:1 to less than 2:1, from 1.75:1 to 1.99:1, from 1.75:1 to 1.95:1, from 1.75:1 to 1.9:1, from 1.75:1 to 1.85:1, from 1.75:1 to 1.8:1, from 1.8:1 to less than 2:1, from 1.8:1 to 1.99:1, from 1.8:1 to 1.95:1, from 1.8:1 to 1.9:1, from 1.8:1 to 1.85:1, from 1.85:1 to less than 2:1, from 1.85:1 to 1.99:1, from 1.85:1 to 1.95:1, from 1.85:1 to 1.9:1, from 1.9:1 to less than 2:1, from 1.9:1 to 1.99:1, from 1.9:1 to 1.95:1, from 1.95:1 to less than 2:1, from 1.95:1 to 1.99:1, or from 1.99:1 to less than 2:1.

In certain embodiments, said reactive distillation may result in removing volatile components, such as water, from the intermediate reaction mixture. For example, but without limitation, if formalin is used, it may be desirable to remove water and methanol (which may be present in the formalin as a stabilizer).

In the context of the phrase “the predominant compound present in the reaction product”, the term “predominant” refers to the compound which is present in the reaction product in an amount higher than any other single compound in the reaction product. In certain embodiments, the 3,3′-methylenebis[5-methyloxazolidine] is present in the reaction product in an amount of at least 30 mole percent. In certain embodiments, the 3,3′-methylenebis[5-methyloxazolidine] is present in the reaction product in an amount of at least 32 mole percent. In certain embodiments, the 3,3′-methylenebis[5-methyloxazolidine] is present in the reaction product in an amount of at least 34 mole percent. In certain embodiments, the 3,3′-methylenebis[5-methyloxazolidine] is present in the reaction product in an amount of at least 36 mole percent. In certain embodiments, the 3,3′-methylenebis[5-methyloxazolidine] is present in the reaction product in an amount of at least 38 mole percent. In certain embodiments, the 3,3′-methylenebis[5-methyloxazolidine] is present in the reaction product in an amount of at least 40 mole percent. In certain embodiments, the 3,3′-methylenebis[5-methyloxazolidine] is present in the reaction product in an amount of at least 42 mole percent. In certain embodiments, the 3,3′-methylenebis[5-methyloxazolidine] is present in the reaction product in an amount of at least 44 mole percent. In certain embodiments, the 3,3′-methylenebis[5-methyloxazolidine] is present in the reaction product in an amount of at least 46 mole percent. In certain embodiments, the 3,3′-methylenebis[5-methyloxazolidine] is present in the reaction product in an amount of at least 48 mole percent. In certain embodiments, the 3,3′-methylenebis[5-methyloxazolidine] is present in the reaction product in an amount of at least 50 mole percent. In certain embodiments, the 3,3′-methylenebis[5-methyloxazolidine] is present in the reaction product in an amount of from 30 to 75 mole percent. In certain embodiments, the 3,3′-methylenebis[5-methyloxazolidine] is present in the reaction product in an amount of from 32 to 75 mole percent. In certain embodiments, the 3,3′-methylenebis[5-methyloxazolidine] is present in the reaction product in an amount of from 34 to 75 mole percent. In certain embodiments, the 3,3′-methylenebis[5-methyloxazolidine] is present in the reaction product in an amount of from 36 to 75 mole percent. In certain embodiments, the 3,3′-methylenebis[5-methyloxazolidine] is present in the reaction product in an amount of from 38 to 75 mole percent. In certain embodiments, the 3,3′-methylenebis[5-methyloxazolidine] is present in the reaction product in an amount of from 40 to 75 mole percent. In certain embodiments, the 3,3′-methylenebis[5-methyloxazolidine] is present in the reaction product in an amount of from 42 to 75 mole percent. In certain embodiments, the 3,3′-methylenebis[5-methyloxazolidine] is present in the reaction product in an amount of from 44 to 75 mole percent. In certain embodiments, the 3,3′-methylenebis[5-methyloxazolidine] is present in the reaction product in an amount of from 46 to 75 mole percent. In certain embodiments, the 3,3′-methylenebis[5-methyloxazolidine] is present in the reaction product in an amount of from 48 to 75 mole percent. In certain embodiments, the 3,3′-methylenebis[5-methyloxazolidine] is present in the reaction product in an amount of from 50 to 75 mole percent. In certain embodiments, the 3,3′-methylenebis[5-methyloxazolidine] is present in the reaction product in an amount of from 52 to 75 mole percent. In certain embodiments, the 3,3′-methylenebis[5-methyloxazolidine] is present in the reaction product in an amount of from 54 to 75 mole percent. In certain embodiments, the 3,3′-methylenebis[5-methyloxazolidine] is present in the reaction product in an amount of from 56 to 75 mole percent. In certain embodiments, the 3,3′-methylenebis[5-methyloxazolidine] is present in the reaction product in an amount of from 58 to 75 mole percent. In certain embodiments, the 3,3′-methylenebis[5-methyloxazolidine] is present in the reaction product in an amount of from 60 to 75 mole percent. In certain embodiments, the 3,3′-methylenebis[5-methyloxazolidine] is present in the reaction product in an amount of from 62 to 75 mole percent. In certain embodiments, the 3,3′-methylenebis[5-methyloxazolidine] is present in the reaction product in an amount of from 64 to 75 mole percent. In certain embodiments, the 3,3′-methylenebis[5-methyloxazolidine] is present in the reaction product in an amount of from 66 to 75 mole percent. In certain embodiments, the 3,3′-methylenebis[5-methyloxazolidine] is present in the reaction product in an amount of from 68 to 75 mole percent. In certain embodiments, the 3,3′-methylenebis[5-methyloxazolidine] is present in the reaction product in an amount of from 70 to 75 mole percent. In certain embodiments, the 3,3′-methylenebis[5-methyloxazolidine] is present in the reaction product in an amount of from 72 to 75 mole percent. In certain embodiments, the 3,3′-methylenebis[5-methyloxazolidine] is present in the reaction product in an amount of from 74 to 75 mole percent. In certain embodiments, the 3,3′-methylenebis[5-methyloxazolidine] is present in the reaction product in an amount of from 30 to 74 mole percent. In certain embodiments, the 3,3′-methylenebis[5-methyloxazolidine] is present in the reaction product in an amount of from 32 to 74 mole percent. In certain embodiments, the 3,3′-methylenebis[5-methyloxazolidine] is present in the reaction product in an amount of from 34 to 74 mole percent. In certain embodiments, the 3,3′-methylenebis[5-methyloxazolidine] is present in the reaction product in an amount of from 36 to 74 mole percent. In certain embodiments, the 3,3′-methylenebis[5-methyloxazolidine] is present in the reaction product in an amount of from 38 to 74 mole percent. In certain embodiments, the 3,3′-methylenebis[5-methyloxazolidine] is present in the reaction product in an amount of from 40 to 74 mole percent. In certain embodiments, the 3,3′-methylenebis[5-methyloxazolidine] is present in the reaction product in an amount of from 42 to 74 mole percent. In certain embodiments, the 3,3′-methylenebis[5-methyloxazolidine] is present in the reaction product in an amount of from 44 to 74 mole percent. In certain embodiments, the 3,3′-methylenebis[5-methyloxazolidine] is present in the reaction product in an amount of from 46 to 74 mole percent. In certain embodiments, the 3,3′-methylenebis[5-methyloxazolidine] is present in the reaction product in an amount of from 48 to 74 mole percent. In certain embodiments, the 3,3′-methylenebis[5-methyloxazolidine] is present in the reaction product in an amount of from 50 to 74 mole percent. In certain embodiments, the 3,3′-methylenebis[5-methyloxazolidine] is present in the reaction product in an amount of from 52 to 74 mole percent. In certain embodiments, the 3,3′-methylenebis[5-methyloxazolidine] is present in the reaction product in an amount of from 54 to 74 mole percent. In certain embodiments, the 3,3′-methylenebis[5-methyloxazolidine] is present in the reaction product in an amount of from 56 to 74 mole percent. In certain embodiments, the 3,3′-methylenebis[5-methyloxazolidine] is present in the reaction product in an amount of from 58 to 74 mole percent. In certain embodiments, the 3,3′-methylenebis[5-methyloxazolidine] is present in the reaction product in an amount of from 60 to 74 mole percent. In certain embodiments, the 3,3′-methylenebis[5-methyloxazolidine] is present in the reaction product in an amount of from 62 to 74 mole percent. In certain embodiments, the 3,3′-methylenebis[5-methyloxazolidine] is present in the reaction product in an amount of from 64 to 74 mole percent. In certain embodiments, the 3,3′-methylenebis[5-methyloxazolidine] is present in the reaction product in an amount of from 66 to 74 mole percent. In certain embodiments, the 3,3′-methylenebis[5-methyloxazolidine] is present in the reaction product in an amount of from 68 to 74 mole percent. In certain embodiments, the 3,3′-methylenebis[5-methyloxazolidine] is present in the reaction product in an amount of from 70 to 74 mole percent. In certain embodiments, the 3,3′-methylenebis[5-methyloxazolidine] is present in the reaction product in an amount of from 72 to 74 mole percent. In certain embodiments, the 3,3′-methylenebis[5-methyloxazolidine] is present in the reaction product in an amount of from 30 to 72 mole percent. In certain embodiments, the 3,3′-methylenebis[5-methyloxazolidine] is present in the reaction product in an amount of from 32 to 72 mole percent. In certain embodiments, the 3,3′-methylenebis[5-methyloxazolidine] is present in the reaction product in an amount of from 34 to 72 mole percent. In certain embodiments, the 3,3′-methylenebis[5-methyloxazolidine] is present in the reaction product in an amount of from 36 to 72 mole percent. In certain embodiments, the 3,3′-methylenebis[5-methyloxazolidine] is present in the reaction product in an amount of from 38 to 72 mole percent. In certain embodiments, the 3,3′-methylenebis[5-methyloxazolidine] is present in the reaction product in an amount of from 40 to 72 mole percent. In certain embodiments, the 3,3′-methylenebis[5-methyloxazolidine] is present in the reaction product in an amount of from 42 to 72 mole percent. In certain embodiments, the 3,3′-methylenebis[5-methyloxazolidine] is present in the reaction product in an amount of from 44 to 72 mole percent. In certain embodiments, the 3,3′-methylenebis[5-methyloxazolidine] is present in the reaction product in an amount of from 46 to 72 mole percent. In certain embodiments, the 3,3′-methylenebis[5-methyloxazolidine] is present in the reaction product in an amount of from 48 to 72 mole percent. In certain embodiments, the 3,3′-methylenebis[5-methyloxazolidine] is present in the reaction product in an amount of from 50 to 72 mole percent. In certain embodiments, the 3,3′-methylenebis[5-methyloxazolidine] is present in the reaction product in an amount of from 52 to 72 mole percent. In certain embodiments, the 3,3′-methylenebis[5-methyloxazolidine] is present in the reaction product in an amount of from 54 to 72 mole percent. In certain embodiments, the 3,3′-methylenebis[5-methyloxazolidine] is present in the reaction product in an amount of from 56 to 72 mole percent. In certain embodiments, the 3,3′-methylenebis[5-methyloxazolidine] is present in the reaction product in an amount of from 58 to 72 mole percent. In certain embodiments, the 3,3′-methylenebis[5-methyloxazolidine] is present in the reaction product in an amount of from 60 to 72 mole percent. In certain embodiments, the 3,3′-methylenebis[5-methyloxazolidine] is present in the reaction product in an amount of from 62 to 72 mole percent. In certain embodiments, the 3,3′-methylenebis[5-methyloxazolidine] is present in the reaction product in an amount of from 64 to 72 mole percent. In certain embodiments, the 3,3′-methylenebis[5-methyloxazolidine] is present in the reaction product in an amount of from 66 to 72 mole percent. In certain embodiments, the 3,3′-methylenebis[5-methyloxazolidine] is present in the reaction product in an amount of from 68 to 72 mole percent. In certain embodiments, the 3,3′-methylenebis[5-methyloxazolidine] is present in the reaction product in an amount of from 70 to 72 mole percent. In certain embodiments, the 3,3′-methylenebis[5-methyloxazolidine] is present in the reaction product in an amount of from 30 to 70 mole percent. In certain embodiments, the 3,3′-methylenebis[5-methyloxazolidine] is present in the reaction product in an amount of from 32 to 70 mole percent. In certain embodiments, the 3,3′-methylenebis[5-methyloxazolidine] is present in the reaction product in an amount of from 34 to 70 mole percent. In certain embodiments, the 3,3′-methylenebis[5-methyloxazolidine] is present in the reaction product in an amount of from 36 to 70 mole percent. In certain embodiments, the 3,3′-methylenebis[5-methyloxazolidine] is present in the reaction product in an amount of from 38 to 70 mole percent. In certain embodiments, the 3,3′-methylenebis[5-methyloxazolidine] is present in the reaction product in an amount of from 40 to 70 mole percent. In certain embodiments, the 3,3′-methylenebis[5-methyloxazolidine] is present in the reaction product in an amount of from 42 to 70 mole percent. In certain embodiments, the 3,3′-methylenebis[5-methyloxazolidine] is present in the reaction product in an amount of from 44 to 70 mole percent. In certain embodiments, the 3,3′-methylenebis[5-methyloxazolidine] is present in the reaction product in an amount of from 46 to 70 mole percent. In certain embodiments, the 3,3′-methylenebis[5-methyloxazolidine] is present in the reaction product in an amount of from 48 to 70 mole percent. In certain embodiments, the 3,3′-methylenebis[5-methyloxazolidine] is present in the reaction product in an amount of from 50 to 70 mole percent. In certain embodiments, the 3,3′-methylenebis[5-methyloxazolidine] is present in the reaction product in an amount of from 52 to 70 mole percent. In certain embodiments, the 3,3′-methylenebis[5-methyloxazolidine] is present in the reaction product in an amount of from 54 to 70 mole percent. In certain embodiments, the 3,3′-methylenebis[5-methyloxazolidine] is present in the reaction product in an amount of from 56 to 70 mole percent. In certain embodiments, the 3,3′-methylenebis[5-methyloxazolidine] is present in the reaction product in an amount of from 58 to 70 mole percent. In certain embodiments, the 3,3′-methylenebis[5-methyloxazolidine] is present in the reaction product in an amount of from 60 to 70 mole percent. In certain embodiments, the 3,3′-methylenebis[5-methyloxazolidine] is present in the reaction product in an amount of from 62 to 70 mole percent. In certain embodiments, the 3,3′-methylenebis[5-methyloxazolidine] is present in the reaction product in an amount of from 64 to 70 mole percent. In certain embodiments, the 3,3′-methylenebis[5-methyloxazolidine] is present in the reaction product in an amount of from 66 to 70 mole percent. In certain embodiments, the 3,3′-methylenebis[5-methyloxazolidine] is present in the reaction product in an amount of from 68 to 70 mole percent. In certain embodiments, the 3,3′-methylenebis[5-methyloxazolidine] is present in the reaction product in an amount of from 30 to 68 mole percent. In certain embodiments, the 3,3′-methylenebis[5-methyloxazolidine] is present in the reaction product in an amount of from 32 to 68 mole percent. In certain embodiments, the 3,3′-methylenebis[5-methyloxazolidine] is present in the reaction product in an amount of from 34 to 68 mole percent. In certain embodiments, the 3,3′-methylenebis[5-methyloxazolidine] is present in the reaction product in an amount of from 36 to 68 mole percent. In certain embodiments, the 3,3′-methylenebis[5-methyloxazolidine] is present in the reaction product in an amount of from 38 to 68 mole percent. In certain embodiments, the 3,3′-methylenebis[5-methyloxazolidine] is present in the reaction product in an amount of from 40 to 68 mole percent. In certain embodiments, the 3,3′-methylenebis[5-methyloxazolidine] is present in the reaction product in an amount of from 42 to 68 mole percent. In certain embodiments, the 3,3′-methylenebis[5-methyloxazolidine] is present in the reaction product in an amount of from 44 to 68 mole percent. In certain embodiments, the 3,3′-methylenebis[5-methyloxazolidine] is present in the reaction product in an amount of from 46 to 68 mole percent. In certain embodiments, the 3,3′-methylenebis[5-methyloxazolidine] is present in the reaction product in an amount of from 48 to 68 mole percent. In certain embodiments, the 3,3′-methylenebis[5-methyloxazolidine] is present in the reaction product in an amount of from 50 to 68 mole percent. In certain embodiments, the 3,3′-methylenebis[5-methyloxazolidine] is present in the reaction product in an amount of from 52 to 68 mole percent. In certain embodiments, the 3,3′-methylenebis[5-methyloxazolidine] is present in the reaction product in an amount of from 54 to 68 mole percent. In certain embodiments, the 3,3′-methylenebis[5-methyloxazolidine] is present in the reaction product in an amount of from 56 to 68 mole percent. In certain embodiments, the 3,3′-methylenebis[5-methyloxazolidine] is present in the reaction product in an amount of from 58 to 68 mole percent. In certain embodiments, the 3,3′-methylenebis[5-methyloxazolidine] is present in the reaction product in an amount of from 60 to 68 mole percent. In certain embodiments, the 3,3′-methylenebis[5-methyloxazolidine] is present in the reaction product in an amount of from 62 to 68 mole percent. In certain embodiments, the 3,3′-methylenebis[5-methyloxazolidine] is present in the reaction product in an amount of from 64 to 68 mole percent. In certain embodiments, the 3,3′-methylenebis[5-methyloxazolidine] is present in the reaction product in an amount of from 66 to 68 mole percent. In certain embodiments, the 3,3′-methylenebis[5-methyloxazolidine] is present in the reaction product in an amount of from 30 to 66 mole percent. In certain embodiments, the 3,3′-methylenebis[5-methyloxazolidine] is present in the reaction product in an amount of from 32 to 66 mole percent. In certain embodiments, the 3,3′-methylenebis[5-methyloxazolidine] is present in the reaction product in an amount of from 34 to 66 mole percent. In certain embodiments, the 3,3′-methylenebis[5-methyloxazolidine] is present in the reaction product in an amount of from 36 to 66 mole percent. In certain embodiments, the 3,3′-methylenebis[5-methyloxazolidine] is present in the reaction product in an amount of from 38 to 66 mole percent. In certain embodiments, the 3,3′-methylenebis[5-methyloxazolidine] is present in the reaction product in an amount of from 40 to 66 mole percent. In certain embodiments, the 3,3′-methylenebis[5-methyloxazolidine] is present in the reaction product in an amount of from 42 to 66 mole percent. In certain embodiments, the 3,3′-methylenebis[5-methyloxazolidine] is present in the reaction product in an amount of from 44 to 66 mole percent. In certain embodiments, the 3,3′-methylenebis[5-methyloxazolidine] is present in the reaction product in an amount of from 46 to 66 mole percent. In certain embodiments, the 3,3′-methylenebis[5-methyloxazolidine] is present in the reaction product in an amount of from 48 to 66 mole percent. In certain embodiments, the 3,3′-methylenebis[5-methyloxazolidine] is present in the reaction product in an amount of from 50 to 66 mole percent. In certain embodiments, the 3,3′-methylenebis[5-methyloxazolidine] is present in the reaction product in an amount of from 52 to 66 mole percent. In certain embodiments, the 3,3′-methylenebis[5-methyloxazolidine] is present in the reaction product in an amount of from 54 to 66 mole percent. In certain embodiments, the 3,3′-methylenebis[5-methyloxazolidine] is present in the reaction product in an amount of from 56 to 66 mole percent. In certain embodiments, the 3,3′-methylenebis[5-methyloxazolidine] is present in the reaction product in an amount of from 58 to 66 mole percent. In certain embodiments, the 3,3′-methylenebis[5-methyloxazolidine] is present in the reaction product in an amount of from 60 to 66 mole percent. In certain embodiments, the 3,3′-methylenebis[5-methyloxazolidine] is present in the reaction product in an amount of from 62 to 66 mole percent. In certain embodiments, the 3,3′-methylenebis[5-methyloxazolidine] is present in the reaction product in an amount of from 64 to 66 mole percent. In certain embodiments, the 3,3′-methylenebis[5-methyloxazolidine] is present in the reaction product in an amount of from 30 to 64 mole percent. In certain embodiments, the 3,3′-methylenebis[5-methyloxazolidine] is present in the reaction product in an amount of from 32 to 64 mole percent. In certain embodiments, the 3,3′-methylenebis[5-methyloxazolidine] is present in the reaction product in an amount of from 34 to 64 mole percent. In certain embodiments, the 3,3′-methylenebis[5-methyloxazolidine] is present in the reaction product in an amount of from 36 to 64 mole percent. In certain embodiments, the 3,3′-methylenebis[5-methyloxazolidine] is present in the reaction product in an amount of from 38 to 64 mole percent. In certain embodiments, the 3,3′-methylenebis[5-methyloxazolidine] is present in the reaction product in an amount of from 40 to 64 mole percent. In certain embodiments, the 3,3′-methylenebis[5-methyloxazolidine] is present in the reaction product in an amount of from 42 to 64 mole percent. In certain embodiments, the 3,3′-methylenebis[5-methyloxazolidine] is present in the reaction product in an amount of from 44 to 64 mole percent. In certain embodiments, the 3,3′-methylenebis[5-methyloxazolidine] is present in the reaction product in an amount of from 46 to 64 mole percent. In certain embodiments, the 3,3′-methylenebis[5-methyloxazolidine] is present in the reaction product in an amount of from 48 to 64 mole percent. In certain embodiments, the 3,3′-methylenebis[5-methyloxazolidine] is present in the reaction product in an amount of from 50 to 64 mole percent. In certain embodiments, the 3,3′-methylenebis[5-methyloxazolidine] is present in the reaction product in an amount of from 52 to 64 mole percent. In certain embodiments, the 3,3′-methylenebis[5-methyloxazolidine] is present in the reaction product in an amount of from 54 to 64 mole percent. In certain embodiments, the 3,3′-methylenebis[5-methyloxazolidine] is present in the reaction product in an amount of from 56 to 64 mole percent. In certain embodiments, the 3,3′-methylenebis[5-methyloxazolidine] is present in the reaction product in an amount of from 58 to 64 mole percent. In certain embodiments, the 3,3′-methylenebis[5-methyloxazolidine] is present in the reaction product in an amount of from 60 to 64 mole percent. In certain embodiments, the 3,3′-methylenebis[5-methyloxazolidine] is present in the reaction product in an amount of from 62 to 64 mole percent. In certain embodiments, the 3,3′-methylenebis[5-methyloxazolidine] is present in the reaction product in an amount of from 30 to 62 mole percent. In certain embodiments, the 3,3′-methylenebis[5-methyloxazolidine] is present in the reaction product in an amount of from 32 to 62 mole percent. In certain embodiments, the 3,3′-methylenebis[5-methyloxazolidine] is present in the reaction product in an amount of from 34 to 62 mole percent. In certain embodiments, the 3,3′-methylenebis[5-methyloxazolidine] is present in the reaction product in an amount of from 36 to 62 mole percent. In certain embodiments, the 3,3′-methylenebis[5-methyloxazolidine] is present in the reaction product in an amount of from 38 to 62 mole percent. In certain embodiments, the 3,3′-methylenebis[5-methyloxazolidine] is present in the reaction product in an amount of from 40 to 62 mole percent. In certain embodiments, the 3,3′-methylenebis[5-methyloxazolidine] is present in the reaction product in an amount of from 42 to 62 mole percent. In certain embodiments, the 3,3′-methylenebis[5-methyloxazolidine] is present in the reaction product in an amount of from 44 to 62 mole percent. In certain embodiments, the 3,3′-methylenebis[5-methyloxazolidine] is present in the reaction product in an amount of from 46 to 62 mole percent. In certain embodiments, the 3,3′-methylenebis[5-methyloxazolidine] is present in the reaction product in an amount of from 48 to 62 mole percent. In certain embodiments, the 3,3′-methylenebis[5-methyloxazolidine] is present in the reaction product in an amount of from 50 to 62 mole percent. In certain embodiments, the 3,3′-methylenebis[5-methyloxazolidine] is present in the reaction product in an amount of from 52 to 62 mole percent. In certain embodiments, the 3,3′-methylenebis[5-methyloxazolidine] is present in the reaction product in an amount of from 54 to 62 mole percent. In certain embodiments, the 3,3′-methylenebis[5-methyloxazolidine] is present in the reaction product in an amount of from 56 to 62 mole percent. In certain embodiments, the 3,3′-methylenebis[5-methyloxazolidine] is present in the reaction product in an amount of from 58 to 62 mole percent. In certain embodiments, the 3,3′-methylenebis[5-methyloxazolidine] is present in the reaction product in an amount of from 60 to 62 mole percent. In certain embodiments, the 3,3′-methylenebis[5-methyloxazolidine] is present in the reaction product in an amount of from 30 to 60 mole percent. In certain embodiments, the 3,3′-methylenebis[5-methyloxazolidine] is present in the reaction product in an amount of from 32 to 60 mole percent. In certain embodiments, the 3,3′-methylenebis[5-methyloxazolidine] is present in the reaction product in an amount of from 34 to 60 mole percent. In certain embodiments, the 3,3′-methylenebis[5-methyloxazolidine] is present in the reaction product in an amount of from 36 to 60 mole percent. In certain embodiments, the 3,3′-methylenebis[5-methyloxazolidine] is present in the reaction product in an amount of from 38 to 60 mole percent. In certain embodiments, the 3,3′-methylenebis[5-methyloxazolidine] is present in the reaction product in an amount of from 40 to 60 mole percent. In certain embodiments, the 3,3′-methylenebis[5-methyloxazolidine] is present in the reaction product in an amount of from 42 to 60 mole percent. In certain embodiments, the 3,3′-methylenebis[5-methyloxazolidine] is present in the reaction product in an amount of from 44 to 60 mole percent. In certain embodiments, the 3,3′-methylenebis[5-methyloxazolidine] is present in the reaction product in an amount of from 46 to 60 mole percent. In certain embodiments, the 3,3′-methylenebis[5-methyloxazolidine] is present in the reaction product in an amount of from 48 to 60 mole percent. In certain embodiments, the 3,3′-methylenebis[5-methyloxazolidine] is present in the reaction product in an amount of from 50 to 60 mole percent. In certain embodiments, the 3,3′-methylenebis[5-methyloxazolidine] is present in the reaction product in an amount of from 52 to 60 mole percent. In certain embodiments, the 3,3′-methylenebis[5-methyloxazolidine] is present in the reaction product in an amount of from 54 to 60 mole percent. In certain embodiments, the 3,3′-methylenebis[5-methyloxazolidine] is present in the reaction product in an amount of from 56 to 60 mole percent. In certain embodiments, the 3,3′-methylenebis[5-methyloxazolidine] is present in the reaction product in an amount of from 58 to 60 mole percent. In certain embodiments, the 3,3′-methylenebis[5-methyloxazolidine] is present in the reaction product in an amount of from 30 to 58 mole percent. In certain embodiments, the 3,3′-methylenebis[5-methyloxazolidine] is present in the reaction product in an amount of from 32 to 58 mole percent. In certain embodiments, the 3,3′-methylenebis[5-methyloxazolidine] is present in the reaction product in an amount of from 34 to 58 mole percent. In certain embodiments, the 3,3′-methylenebis[5-methyloxazolidine] is present in the reaction product in an amount of from 36 to 58 mole percent. In certain embodiments, the 3,3′-methylenebis[5-methyloxazolidine] is present in the reaction product in an amount of from 38 to 58 mole percent. In certain embodiments, the 3,3′-methylenebis[5-methyloxazolidine] is present in the reaction product in an amount of from 40 to 58 mole percent. In certain embodiments, the 3,3′-methylenebis[5-methyloxazolidine] is present in the reaction product in an amount of from 42 to 58 mole percent. In certain embodiments, the 3,3′-methylenebis[5-methyloxazolidine] is present in the reaction product in an amount of from 44 to 58 mole percent. In certain embodiments, the 3,3′-methylenebis[5-methyloxazolidine] is present in the reaction product in an amount of from 46 to 58 mole percent. In certain embodiments, the 3,3′-methylenebis[5-methyloxazolidine] is present in the reaction product in an amount of from 48 to 58 mole percent. In certain embodiments, the 3,3′-methylenebis[5-methyloxazolidine] is present in the reaction product in an amount of from 50 to 58 mole percent. In certain embodiments, the 3,3′-methylenebis[5-methyloxazolidine] is present in the reaction product in an amount of from 52 to 58 mole percent. In certain embodiments, the 3,3′-methylenebis[5-methyloxazolidine] is present in the reaction product in an amount of from 54 to 58 mole percent. In certain embodiments, the 3,3′-methylenebis[5-methyloxazolidine] is present in the reaction product in an amount of from 56 to 58 mole percent. In certain embodiments, the 3,3′-methylenebis[5-methyloxazolidine] is present in the reaction product in an amount of from 30 to 56 mole percent. In certain embodiments, the 3,3′-methylenebis[5-methyloxazolidine] is present in the reaction product in an amount of from 32 to 56 mole percent. In certain embodiments, the 3,3′-methylenebis[5-methyloxazolidine] is present in the reaction product in an amount of from 34 to 56 mole percent. In certain embodiments, the 3,3′-methylenebis[5-methyloxazolidine] is present in the reaction product in an amount of from 36 to 56 mole percent. In certain embodiments, the 3,3′-methylenebis[5-methyloxazolidine] is present in the reaction product in an amount of from 38 to 56 mole percent. In certain embodiments, the 3,3′-methylenebis[5-methyloxazolidine] is present in the reaction product in an amount of from 40 to 56 mole percent. In certain embodiments, the 3,3′-methylenebis[5-methyloxazolidine] is present in the reaction product in an amount of from 42 to 56 mole percent. In certain embodiments, the 3,3′-methylenebis[5-methyloxazolidine] is present in the reaction product in an amount of from 44 to 56 mole percent. In certain embodiments, the 3,3′-methylenebis[5-methyloxazolidine] is present in the reaction product in an amount of from 46 to 56 mole percent. In certain embodiments, the 3,3′-methylenebis[5-methyloxazolidine] is present in the reaction product in an amount of from 48 to 56 mole percent. In certain embodiments, the 3,3′-methylenebis[5-methyloxazolidine] is present in the reaction product in an amount of from 50 to 56 mole percent. In certain embodiments, the 3,3′-methylenebis[5-methyloxazolidine] is present in the reaction product in an amount of from 52 to 56 mole percent. In certain embodiments, the 3,3′-methylenebis[5-methyloxazolidine] is present in the reaction product in an amount of from 54 to 56 mole percent. In certain embodiments, the 3,3′-methylenebis[5-methyloxazolidine] is present in the reaction product in an amount of from 30 to 54 mole percent. In certain embodiments, the 3,3′-methylenebis[5-methyloxazolidine] is present in the reaction product in an amount of from 32 to 54 mole percent. In certain embodiments, the 3,3′-methylenebis[5-methyloxazolidine] is present in the reaction product in an amount of from 34 to 54 mole percent. In certain embodiments, the 3,3′-methylenebis[5-methyloxazolidine] is present in the reaction product in an amount of from 36 to 54 mole percent. In certain embodiments, the 3,3′-methylenebis[5-methyloxazolidine] is present in the reaction product in an amount of from 38 to 54 mole percent. In certain embodiments, the 3,3′-methylenebis[5-methyloxazolidine] is present in the reaction product in an amount of from 40 to 54 mole percent. In certain embodiments, the 3,3′-methylenebis[5-methyloxazolidine] is present in the reaction product in an amount of from 42 to 54 mole percent. In certain embodiments, the 3,3′-methylenebis[5-methyloxazolidine] is present in the reaction product in an amount of from 44 to 54 mole percent. In certain embodiments, the 3,3′-methylenebis[5-methyloxazolidine] is present in the reaction product in an amount of from 46 to 54 mole percent. In certain embodiments, the 3,3′-methylenebis[5-methyloxazolidine] is present in the reaction product in an amount of from 48 to 54 mole percent. In certain embodiments, the 3,3′-methylenebis[5-methyloxazolidine] is present in the reaction product in an amount of from 50 to 54 mole percent. In certain embodiments, the 3,3′-methylenebis[5-methyloxazolidine] is present in the reaction product in an amount of from 52 to 54 mole percent. In certain embodiments, the 3,3′-methylenebis[5-methyloxazolidine] is present in the reaction product in an amount of from 30 to 52 mole percent. In certain embodiments, the 3,3′-methylenebis[5-methyloxazolidine] is present in the reaction product in an amount of from 32 to 52 mole percent. In certain embodiments, the 3,3′-methylenebis[5-methyloxazolidine] is present in the reaction product in an amount of from 34 to 52 mole percent. In certain embodiments, the 3,3′-methylenebis[5-methyloxazolidine] is present in the reaction product in an amount of from 36 to 52 mole percent. In certain embodiments, the 3,3′-methylenebis[5-methyloxazolidine] is present in the reaction product in an amount of from 38 to 52 mole percent. In certain embodiments, the 3,3′-methylenebis[5-methyloxazolidine] is present in the reaction product in an amount of from 40 to 52 mole percent. In certain embodiments, the 3,3′-methylenebis[5-methyloxazolidine] is present in the reaction product in an amount of from 42 to 52 mole percent. In certain embodiments, the 3,3′-methylenebis[5-methyloxazolidine] is present in the reaction product in an amount of from 44 to 52 mole percent. In certain embodiments, the 3,3′-methylenebis[5-methyloxazolidine] is present in the reaction product in an amount of from 46 to 52 mole percent. In certain embodiments, the 3,3′-methylenebis[5-methyloxazolidine] is present in the reaction product in an amount of from 48 to 52 mole percent. In certain embodiments, the 3,3′-methylenebis[5-methyloxazolidine] is present in the reaction product in an amount of from 50 to 52 mole percent. In certain embodiments, the 3,3′-methylenebis[5-methyloxazolidine] is present in the reaction product in an amount of from 30 to 50 mole percent. In certain embodiments, the 3,3′-methylenebis[5-methyloxazolidine] is present in the reaction product in an amount of from 32 to 50 mole percent. In certain embodiments, the 3,3′-methylenebis[5-methyloxazolidine] is present in the reaction product in an amount of from 34 to 50 mole percent. In certain embodiments, the 3,3′-methylenebis[5-methyloxazolidine] is present in the reaction product in an amount of from 36 to 50 mole percent. In certain embodiments, the 3,3′-methylenebis[5-methyloxazolidine] is present in the reaction product in an amount of from 38 to 50 mole percent. In certain embodiments, the 3,3′-methylenebis[5-methyloxazolidine] is present in the reaction product in an amount of from 40 to 50 mole percent. In certain embodiments, the 3,3′-methylenebis[5-methyloxazolidine] is present in the reaction product in an amount of from 42 to 50 mole percent. In certain embodiments, the 3,3′-methylenebis[5-methyloxazolidine] is present in the reaction product in an amount of from 44 to 50 mole percent. In certain embodiments, the 3,3′-methylenebis[5-methyloxazolidine] is present in the reaction product in an amount of from 46 to 50 mole percent. In certain embodiments, the 3,3′-methylenebis[5-methyloxazolidine] is present in the reaction product in an amount of from 48 to 50 mole percent. In certain embodiments, the 3,3′-methylenebis[5-methyloxazolidine] is present in the reaction product in an amount of from 30 to 48 mole percent. In certain embodiments, the 3,3′-methylenebis[5-methyloxazolidine] is present in the reaction product in an amount of from 32 to 48 mole percent. In certain embodiments, the 3,3′-methylenebis[5-methyloxazolidine] is present in the reaction product in an amount of from 34 to 48 mole percent. In certain embodiments, the 3,3′-methylenebis[5-methyloxazolidine] is present in the reaction product in an amount of from 36 to 48 mole percent. In certain embodiments, the 3,3′-methylenebis[5-methyloxazolidine] is present in the reaction product in an amount of from 38 to 48 mole percent. In certain embodiments, the 3,3′-methylenebis[5-methyloxazolidine] is present in the reaction product in an amount of from 40 to 48 mole percent. In certain embodiments, the 3,3′-methylenebis[5-methyloxazolidine] is present in the reaction product in an amount of from 42 to 48 mole percent. In certain embodiments, the 3,3′-methylenebis[5-methyloxazolidine] is present in the reaction product in an amount of from 44 to 48 mole percent. In certain embodiments, the 3,3′-methylenebis[5-methyloxazolidine] is present in the reaction product in an amount of from 46 to 48 mole percent. In certain embodiments, the 3,3′-methylenebis[5-methyloxazolidine] is present in the reaction product in an amount of from 30 to 46 mole percent. In certain embodiments, the 3,3′-methylenebis[5-methyloxazolidine] is present in the reaction product in an amount of from 32 to 46 mole percent. In certain embodiments, the 3,3′-methylenebis[5-methyloxazolidine] is present in the reaction product in an amount of from 34 to 46 mole percent. In certain embodiments, the 3,3′-methylenebis[5-methyloxazolidine] is present in the reaction product in an amount of from 36 to 46 mole percent. In certain embodiments, the 3,3′-methylenebis[5-methyloxazolidine] is present in the reaction product in an amount of from 38 to 46 mole percent. In certain embodiments, the 3,3′-methylenebis[5-methyloxazolidine] is present in the reaction product in an amount of from 40 to 46 mole percent. In certain embodiments, the 3,3′-methylenebis[5-methyloxazolidine] is present in the reaction product in an amount of from 42 to 46 mole percent. In certain embodiments, the 3,3′-methylenebis[5-methyloxazolidine] is present in the reaction product in an amount of from 44 to 46 mole percent. In certain embodiments, the 3,3′-methylenebis[5-methyloxazolidine] is present in the reaction product in an amount of from 30 to 44 mole percent. In certain embodiments, the 3,3′-methylenebis[5-methyloxazolidine] is present in the reaction product in an amount of from 32 to 44 mole percent. In certain embodiments, the 3,3′-methylenebis[5-methyloxazolidine] is present in the reaction product in an amount of from 34 to 44 mole percent. In certain embodiments, the 3,3′-methylenebis[5-methyloxazolidine] is present in the reaction product in an amount of from 36 to 44 mole percent. In certain embodiments, the 3,3′-methylenebis[5-methyloxazolidine] is present in the reaction product in an amount of from 38 to 44 mole percent. In certain embodiments, the 3,3′-methylenebis[5-methyloxazolidine] is present in the reaction product in an amount of from 40 to 44 mole percent. In certain embodiments, the 3,3′-methylenebis[5-methyloxazolidine] is present in the reaction product in an amount of from 42 to 44 mole percent. In certain embodiments, the 3,3′-methylenebis[5-methyloxazolidine] is present in the reaction product in an amount of from 30 to 42 mole percent. In certain embodiments, the 3,3′-methylenebis[5-methyloxazolidine] is present in the reaction product in an amount of from 32 to 42 mole percent. In certain embodiments, the 3,3′-methylenebis[5-methyloxazolidine] is present in the reaction product in an amount of from 34 to 42 mole percent. In certain embodiments, the 3,3′-methylenebis[5-methyloxazolidine] is present in the reaction product in an amount of from 36 to 42 mole percent. In certain embodiments, the 3,3′-methylenebis[5-methyloxazolidine] is present in the reaction product in an amount of from 38 to 42 mole percent. In certain embodiments, the 3,3′-methylenebis[5-methyloxazolidine] is present in the reaction product in an amount of from 40 to 42 mole percent. In certain embodiments, the 3,3′-methylenebis[5-methyloxazolidine] is present in the reaction product in an amount of from 30 to 40 mole percent. In certain embodiments, the 3,3′-methylenebis[5-methyloxazolidine] is present in the reaction product in an amount of from 32 to 40 mole percent. In certain embodiments, the 3,3′-methylenebis[5-methyloxazolidine] is present in the reaction product in an amount of from 34 to 40 mole percent. In certain embodiments, the 3,3′-methylenebis[5-methyloxazolidine] is present in the reaction product in an amount of from 36 to 40 mole percent. In certain embodiments, the 3,3′-methylenebis[5-methyloxazolidine] is present in the reaction product in an amount of from 38 to 40 mole percent. In certain embodiments, the 3,3′-methylenebis[5-methyloxazolidine] is present in the reaction product in an amount of from 30 to 38 mole percent. In certain embodiments, the 3,3′-methylenebis[5-methyloxazolidine] is present in the reaction product in an amount of from 32 to 38 mole percent. In certain embodiments, the 3,3′-methylenebis[5-methyloxazolidine] is present in the reaction product in an amount of from 34 to 38 mole percent. In certain embodiments, the 3,3′-methylenebis[5-methyloxazolidine] is present in the reaction product in an amount of from 36 to 38 mole percent. In certain embodiments, the 3,3′-methylenebis[5-methyloxazolidine] is present in the reaction product in an amount of from 30 to 36 mole percent. In certain embodiments, the 3,3′-methylenebis[5-methyloxazolidine] is present in the reaction product in an amount of from 32 to 36 mole percent. In certain embodiments, the 3,3′-methylenebis[5-methyloxazolidine] is present in the reaction product in an amount of from 34 to 36 mole percent. In certain embodiments, the 3,3′-methylenebis[5-methyloxazolidine] is present in the reaction product in an amount of from 30 to 34 mole percent. In certain embodiments, the 3,3′-methylenebis[5-methyloxazolidine] is present in the reaction product in an amount of from 32 to 34 mole percent. In certain embodiments, the 3,3′-methylenebis[5-methyloxazolidine] is present in the reaction product in an amount of from 30 to 32 mole percent.

Mixtures of 2,2,4-trimethylpentane (also known as isooctane) and cyclohexane are useful for quick semi-quantitative determination of hydroxyl-rich species such as 1,3,5-tris(2-hydroxypropyl)hexahydro-s-triazine and water in the product. More cyclohexane, relative to isooctane, is needed to completely dissolve the product at room temperature with no visible haze or phase separation as the amounts of hydroxyl-rich species increase (which characterizes a “clear solution” as that term is used in this paragraph). In certain embodiments, the reaction product will give a clear solution when mixed 1:1 by weight with isooctane at room temperature. In certain embodiments, the reaction product will give a clear solution when mixed 1:1 by weight with a 95:5 wt % mixture of isooctane and cyclohexane at room temperature. In certain embodiments, the reaction product will give a clear solution when mixed 1:1 by weight with a 90:10 wt % mixture of isooctane and cyclohexane at room temperature. In certain embodiments, the reaction product will give a clear solution when mixed 1:1 by weight with a 85:15 wt % mixture of isooctane and cyclohexane at room temperature. In certain embodiments, the reaction product will give a clear solution when mixed 1:1 by weight with a 80:20 wt % mixture of isooctane and cyclohexane at room temperature. In certain embodiments, the reaction product will give a clear solution when mixed 1:1 by weight with kerosene at room temperature. In certain embodiments, the reaction product will give a clear solution when mixed 1:1 by weight with diesel fuel at room temperature.

In certain embodiments, the reaction product will have a viscosity of less than 15 cPs at 20° C. and/or less than 175 cPs at −10° C. Viscosity is measured on Brookfield viscometer with a cone-and-plate geometry, 40 mm, 1°, and at a shear rate of 200 s⁻¹ with a temperature sweep from 25° C. down to −40° C.

In certain embodiments, adding the 1-amino-2-propanol into the liquid form of formaldehyde is performed in one or multiple increments at different stages of a continuous reactor. The continuous reactor may contain a baffled internal path to ensure efficient mixing of the reactants and/or an external cooling jacket for temperature control. The addition of 1-amino-2propanol into the liquid form of formaldehyde is performed in a manner that allows enough mixing to ensure that no regions within the reactor have an excess of 1-amino-2-propanol relative to formaldehyde on a molar basis. In certain embodiments, the 1-amino-2-propanol was injected at three equally separated points along the path of the continuous reactor. In certain embodiments, the 1-amino-2-propanol was injected at two equally separated points along the path of the continuous reactor. In certain embodiments, the 1-amino-2-propanol was injected in one single charge into the continuous reactor. In certain embodiments, adding the 1-amino-2-propanol into the liquid form of formaldehyde is performed directly in the distillation column with the liquid form of formaldehyde fed at the bottom of the distillation column, and 1-amino-2-propanol fed at the mid-level of the distillation column. In certain embodiments, adding the 1-amino-2-propanol into the liquid form of formaldehyde is performed in a short pipe, immediately before the resulting mixture is injected into the mid-level of the distillation column. In certain embodiments, adding the 1-amino-2-propanol into the liquid form of formaldehyde is performed at atmospheric pressure while keeping the internal temperature of the reactor at less than 100° C. to prevent the vaporization of the aqueous contents. However, the reactor temperature may be raised to higher temperatures (120° C.) in certain embodiments, as long as the pressure in the reactor is increased sufficiently to maintain all reaction components in their liquid form.

In certain embodiments, said reactive distillation is performed such that there is a sufficient combination of time and temperature to achieve a completed reaction product. Here, the term “reactive distillation” is defined as a thermally driven, vapor-liquid separation process in which a reaction, desired or not, is co-occurring in at least one theoretical stage of a distillation column which may or may not include a reboiler and/or condenser. It is desired that during the reactive distillation the concentration of water in the bottom product stream be reduced sufficiently to enable conversion of any intermediate products to the desired product. In certain embodiments, the water is reduced to less than 20 (such as less than 19, less than 18, less than 17, less than 16, less than 15, less than 14, less than 13, less than 12, less than 11, less than 10, less than 9, less than 8, less than 7, less than 6, less than 5, less than 4.5, less than 4, less than 3.5, less than 3, less than 2.5, less than 2, less than 1.5, less than 1, or less than 0.5) percent by weight, based on the total weight of the reaction product. The water removal may be accomplished through a sufficient combination of temperature and pressure. “Temperature” in this context is defined as the column reboiler temperature. In certain embodiments it is preferable to have a temperature of greater than 80° C., but less than 150° C. In certain embodiments, the temperature is from 80° C. to 150° C., such as from 85° C. to 150° C., from 90° C. to 150° C., from 95° C. to 150° C., from 100° C. to 150° C., from 105° C. to 150° C., from 110° C. to 150° C., from 115° C. to 150° C., from 120° C. to 150° C., from 125° C. to 150° C., from 130° C. to 150° C., from 135° C. to 150° C., from 140° C. to 150° C., from 145° C. to 150° C., from 80° C. to 145° C., 85° C. to 145° C., from 90° C. to 145° C., from 95° C. to 145° C., from 100° C. to 145° C., from 105° C. to 145° C., from 110° C. to 145° C., from 115° C. to 145° C., from 120° C. to 145° C., from 125° C. to 145° C., from 130° C. to 145° C., from 135° C. to 145° C., from 140° C. to 145° C., from 80° C. to 140° C., 85° C. to 140° C., from 90° C. to 140° C., from 95° C. to 140° C., from 100° C. to 140° C., from 105° C. to 140° C., from 110° C. to 140° C., from 115° C. to 140° C., from 120° C. to 140° C., from 125° C. to 140° C., from 130° C. to 140° C., from 135° C. to 140° C., from 80° C. to 135° C., 85° C. to 135° C., from 90° C. to 135° C., from 95° C. to 135° C., from 100° C. to 135° C., from 105° C. to 135° C., from 110° C. to 135° C., from 115° C. to 135° C., from 120° C. to 135° C., from 125° C. to 135° C., from 130° C. to 135° C., from 80° C. to 130° C., 85° C. to 130° C., from 90° C. to 130° C., from 95° C. to 130° C., from 100° C. to 130° C., from 105° C. to 130° C., from 110° C. to 130° C., from 115° C. to 130° C., from 120° C. to 130° C., from 125° C. to 130° C., from 80° C. to 125° C., 85° C. to 125° C., from 90° C. to 125° C., from 95° C. to 125° C., from 100° C. to 125° C., from 105° C. to 125° C., from 110° C. to 125° C., from 115° C. to 125° C., from 120° C. to 125° C., from 80° C. to 120° C., 85° C. to 120° C., from 90° C. to 120° C., from 95° C. to 120° C., from 100° C. to 120° C., from 105° C. to 120° C., from 110° C. to 120° C., from 115° C. to 120° C., from 80° C. to 115° C., 85° C. to 115° C., from 90° C. to 115° C., from 95° C. to 115° C., from 100° C. to 115° C., from 105° C. to 115° C., from 110° C. to 115° C., from 80° C. to 110° C., 85° C. to 110° C., from 90° C. to 110° C., from 95° C. to 110° C., from 100° C. to 110° C., from 105° C. to 110° C., from 80° C. to 105° C., 85° C. to 105° C., from 90° C. to 105° C., from 95° C. to 105° C., from 100° C. to 105° C., from 80° C. to 100° C., 85° C. to 100° C., from 90° C. to 100° C., from 95° C. to 100° C., from 80° C. to 95° C., 85° C. to 95° C., from 90° C. to 95° C., from 80° C. to 90° C., 85° C. to 90° C., or from 80° C. to 85° C. “Pressure” is defined in this context as the absolute pressure at the top of the column just above the top of the theoretical stages. The pressure may be modulated using a vacuum pump to dehydrate the material stream to the desired water content. The pressure required to obtain the desired water content varies based on the temperature of the reboiler for a specific embodiment, and in certain embodiments the pressure is modulated to less than 150 (such as less than 145, less than 140, less than 135, less than 130, less than 125, less than 120, less than 115, less than 110, less than 105, less than 100, less than 95, less than 90, less than 85, less than 80, less than 75, or less than 70) mmHg. In addition to reduction of water content, sufficient residence time of liquid and vapor within the distillation column may be required to allow the desired reaction to complete. In this context “residence time” refers to the amount of time that a mean molecule of reactant in the liquid phase spends within the volume of the distillation column. It is preferable to achieve a residence time between 10 seconds and 15 minutes, more preferably [Vince to insert every residence time range imaginable], and most preferably between 1 and 6 minutes. In certain embodiments, the residence time is from 10 seconds to 15 minutes, such as from 20 seconds to 15 minutes, from 30 seconds to 15 minutes, from 40 seconds to 15 minutes, from 50 seconds to 15 minutes, from 1 minute to 15 minutes, from 2 minutes to 15 minutes, from 3 minutes to 15 minutes, from 4 minutes to 15 minutes, from 5 minutes to 15 minutes, from 6 minutes to 15 minutes, from 7 minutes to 15 minutes, from 8 minutes to 15 minutes, from 9 minutes to 15 minutes, from 10 minutes to 15 minutes, from 11 minutes to 15 minutes, from 12 minutes to 15 minutes, from 13 minutes to 15 minutes, from 14 minutes to 15 minutes, from 10 seconds to 14 minutes, from 20 seconds to 14 minutes, from 30 seconds to 14 minutes, from 40 seconds to 14 minutes, from 50 seconds to 14 minutes, from 1 minute to 14 minutes, from 2 minutes to 14 minutes, from 3 minutes to 14 minutes, from 4 minutes to 14 minutes, from 5 minutes to 14 minutes, from 6 minutes to 14 minutes, from 7 minutes to 14 minutes, from 8 minutes to 14 minutes, from 9 minutes to 14 minutes, from 10 minutes to 14 minutes, from 11 minutes to 14 minutes, from 12 minutes to 14 minutes, from 13 minutes to 14 minutes, from 10 seconds to 13 minutes, from 20 seconds to 13 minutes, from 30 seconds to 13 minutes, from 40 seconds to 13 minutes, from 50 seconds to 13 minutes, from 1 minute to 13 minutes, from 2 minutes to 13 minutes, from 3 minutes to 13 minutes, from 4 minutes to 13 minutes, from 5 minutes to 13 minutes, from 6 minutes to 13 minutes, from 7 minutes to 13 minutes, from 8 minutes to 13 minutes, from 9 minutes to 13 minutes, from 10 minutes to 13 minutes, from 11 minutes to 13 minutes, from 12 minutes to 13 minutes, from 10 seconds to 12 minutes, from 20 seconds to 12 minutes, from 30 seconds to 12 minutes, from 40 seconds to 12 minutes, from 50 seconds to 12 minutes, from 1 minute to 12 minutes, from 2 minutes to 12 minutes, from 3 minutes to 12 minutes, from 4 minutes to 12 minutes, from 5 minutes to 12 minutes, from 6 minutes to 12 minutes, from 7 minutes to 12 minutes, from 8 minutes to 12 minutes, from 9 minutes to 12 minutes, from 10 minutes to 12 minutes, from 11 minutes to 12 minutes, from 10 seconds to 11 minutes, from 20 seconds to 11 minutes, from 30 seconds to 11 minutes, from 40 seconds to 11 minutes, from 50 seconds to 11 minutes, from 1 minute to 11 minutes, from 2 minutes to 11 minutes, from 3 minutes to 11 minutes, from 4 minutes to 11 minutes, from 5 minutes to 11 minutes, from 6 minutes to 11 minutes, from 7 minutes to 11 minutes, from 8 minutes to 11 minutes, from 9 minutes to 11 minutes, from 10 minutes to 11 minutes, from 10 seconds to 10 minutes, from 20 seconds to 10 minutes, from 30 seconds to 10 minutes, from 40 seconds to 10 minutes, from 50 seconds to 10 minutes, from 1 minute to 10 minutes, from 2 minutes to 10 minutes, from 3 minutes to 10 minutes, from 4 minutes to 10 minutes, from 5 minutes to 10 minutes, from 6 minutes to 10 minutes, from 7 minutes to 10 minutes, from 8 minutes to 10 minutes, from 9 minutes to 10 minutes, from 10 seconds to 9 minutes, from 20 seconds to 9 minutes, from 30 seconds to 9 minutes, from 40 seconds to 9 minutes, from 50 seconds to 9 minutes, from 1 minute to 9 minutes, from 2 minutes to 9 minutes, from 3 minutes to 9 minutes, from 4 minutes to 9 minutes, from 5 minutes to 9 minutes, from 6 minutes to 9 minutes, from 7 minutes to 9 minutes, from 8 minutes to 9 minutes, from 10 seconds to 8 minutes, from 20 seconds to 8 minutes, from 30 seconds to 8 minutes, from 40 seconds to 8 minutes, from 50 seconds to 8 minutes, from 1 minute to 8 minutes, from 2 minutes to 8 minutes, from 3 minutes to 8 minutes, from 4 minutes to 8 minutes, from 5 minutes to 8 minutes, from 6 minutes to 8 minutes, from 7 minutes to 8 minutes, from 10 seconds to 7 minutes, from 20 seconds to 7 minutes, from 30 seconds to 7 minutes, from 40 seconds to 7 minutes, from 50 seconds to 7 minutes, from 1 minute to 7 minutes, from 2 minutes to 7 minutes, from 3 minutes to 7 minutes, from 4 minutes to 7 minutes, from 5 minutes to 7 minutes, from 6 minutes to 7 minutes, from 10 seconds to 6 minutes, from 20 seconds to 6 minutes, from 30 seconds to 6 minutes, from 40 seconds to 6 minutes, from 50 seconds to 6 minutes, from 1 minute to 6 minutes, from 2 minutes to 6 minutes, from 3 minutes to 6 minutes, from 4 minutes to 6 minutes, from 5 minutes to 6 minutes, from 10 seconds to 5 minutes, from 20 seconds to 5 minutes, from 30 seconds to 5 minutes, from 40 seconds to 5 minutes, from 50 seconds to 5 minutes, from 1 minute to 5 minutes, from 2 minutes to 5 minutes, from 3 minutes to 5 minutes, from 4 minutes to 5 minutes, from 10 seconds to 4 minutes, from 20 seconds to 4 minutes, from 30 seconds to 4 minutes, from 40 seconds to 4 minutes, from 40 seconds to 4 minutes, from 1 minute to 4 minutes, from 2 minutes to 4 minutes, from 3 minutes to 4 minutes, from 10 seconds to 3 minutes, from 20 seconds to 3 minutes, from 30 seconds to 3 minutes, from 40 seconds to 3 minutes, from 30 seconds to 3 minutes, from 1 minute to 3 minutes, from 2 minutes to 3 minutes, from 10 seconds to 2 minutes, from 20 seconds to 2 minutes, from 30 seconds to 2 minutes, from 40 seconds to 2 minutes, from 20 seconds to 2 minutes, from 1 minute to 2 minutes, from 10 seconds to 1 minute, from 20 seconds to 1 minute, from 30 seconds to 1 minute, from 40 seconds to 1 minute, from 50 seconds to 1 minute, from 10 seconds to 50 seconds, from 20 seconds to 50 seconds, from 30 seconds to 50 seconds, from 40 seconds to 50 seconds, from 10 seconds to 40 seconds, from 20 seconds to 40 seconds, from 30 seconds to 40 seconds, from 10 seconds to 30 seconds, from 20 seconds to 30 seconds, or from 10 seconds to 20 seconds.

In certain embodiments, formation of the intermediate reaction mixture is substantially complete instantaneously after adding the 1-amino-2-propanol into the liquid form of formaldehyde. In this context, the phrase “substantially complete instantaneously” has two facets, and any definitions of the term “substantially” outside of this paragraph do not apply to this phrase. First, the formation is “substantially complete”, meaning that the reaction which forms the intermediate reaction mixture is as complete as it can practically be, such that there could be a small amount of unreacted reactants that will either take a longer than normal time to react, or remain unreacted, but that any such materials would not have any noticeable impact on the performance of the intermediate reaction mixture and/or the reaction product. Second, by “instantaneous”, what is meant is that the reaction which forms the intermediate reaction mixture happens very quickly, such that the speed of the reaction is only practically limited by how quickly the 1-amino-2-propanol diffuses into (and/or is contacted with) the liquid form of formaldehyde; in other words, the reaction occurs instantaneously as molecules of the reactants encounter one another.

In certain embodiments, the reaction product comprises less than 30 mole percent of 1,3,5-tris(2-hydroxypropyl)hexahydro-s-triazine. Reducing the amount of the 1,3,5-tris(2-hydroxypropyl)hexahydro-s-triazine and subsequent increase in mole percentage of 3,3′-methylenebis[5-methyloxazolidine] has the advantage of having a higher formaldehyde molar ratio in the reaction product. Increases in formaldehyde content will increase the capacity of H2S (and/or other sulfur-containing compounds) scavenged and thus when formaldehyde has been added to triazine scavengers there is an increase in the overall uptake in H₂S scavenged versus volume of the product. This direct addition of formaldehyde may present its own set of concerns, as the excess formaldehyde may require additional safety considerations and laboratory testing indicates that solid by-product issues may be increased. The use of the 3,3′-methylenebis[5-methyloxazolidine], which has a ratio of 1:1.5 amine: formaldehyde, versus the 1,3,5-tris(2-hydroxypropyl)hexahydro-s-triazine at 1:1 amine: formaldehyde, gives a higher capacity for H2S consumption potential for 3,3′-methylenebis[5-methyloxazolidine] without the safety concerns of direct additions of formaldehyde.

In certain embodiments, the reaction product comprises less than 28 mole percent of 1,3,5-tris(2-hydroxypropyl)hexahydro-s-triazine. In certain embodiments, the reaction product comprises less than 26 mole percent of 1,3,5-tris(2-hydroxypropyl)hexahydro-s-triazine. In certain embodiments, the reaction product comprises less than 24 mole percent of 1,3,5-tris(2-hydroxypropyl)hexahydro-s-triazine. In certain embodiments, the reaction product comprises less than 22 mole percent of 1,3,5-tris(2-hydroxypropyl)hexahydro-s-triazine. In certain embodiments, the reaction product comprises less than 20 mole percent of 1,3,5-tris (2-hydroxypropyl)hexahydro-s-triazine. In certain embodiments, the reaction product comprises less than 18 mole percent of 1,3,5-tris(2-hydroxypropyl)hexahydro-s-triazine. In certain embodiments, the reaction product comprises less than 16 mole percent of 1,3,5-tris (2-hydroxypropyl)hexahydro-s-triazine. In certain embodiments, the reaction product comprises less than 14 mole percent of 1,3,5-tris(2-hydroxypropyl)hexahydro-s-triazine. In certain embodiments, the reaction product comprises less than 12 mole percent of 1,3,5-tris (2-hydroxypropyl)hexahydro-s-triazine. In certain embodiments, the reaction product comprises less than 10 mole percent of 1,3,5-tris(2-hydroxypropyl)hexahydro-s-triazine. In certain embodiments, the reaction product comprises less than 9 mole percent of 1,3,5-tris (2-hydroxypropyl)hexahydro-s-triazine. In certain embodiments, the reaction product comprises less than 8 mole percent of 1,3,5-tris(2-hydroxypropyl)hexahydro-s-triazine. In certain embodiments, the reaction product comprises less than 7 mole percent of 1,3,5-tris (2-hydroxypropyl)hexahydro-s-triazine. In certain embodiments, the reaction product comprises less than 6 mole percent of 1,3,5-tris(2-hydroxypropyl)hexahydro-s-triazine. In certain embodiments, the reaction product comprises less than 5 mole percent of 1,3,5-tris (2-hydroxypropyl)hexahydro-s-triazine. In certain embodiments, the reaction product comprises less than 4 mole percent of 1,3,5-tris(2-hydroxypropyl)hexahydro-s-triazine. In certain embodiments, the reaction product comprises less than 3 mole percent of 1,3,5-tris (2-hydroxypropyl)hexahydro-s-triazine. In certain embodiments, the reaction product comprises less than 2 mole percent of 1,3,5-tris(2-hydroxypropyl)hexahydro-s-triazine. In certain embodiments, the reaction product comprises less than 1 mole percent of 1,3,5-tris (2-hydroxypropyl)hexahydro-s-triazine. In certain embodiments, the reaction product comprises from greater than 0 to 30 mole percent of 1,3,5-tris(2-hydroxypropyl)hexahydro-s-triazine. In certain embodiments, the reaction product comprises from 1 to 30 mole percent of 1,3,5-tris (2-hydroxypropyl)hexahydro-s-triazine. In certain embodiments, the reaction product comprises from 2 to 30 mole percent of 1,3,5-tris(2-hydroxypropyl)hexahydro-s-triazine. In certain embodiments, the reaction product comprises from 3 to 30 mole percent of 1,3,5-tris (2-hydroxypropyl)hexahydro-s-triazine. In certain embodiments, the reaction product comprises from 4 to 30 mole percent of 1,3,5-tris(2-hydroxypropyl)hexahydro-s-triazine. In certain embodiments, the reaction product comprises from 5 to 30 mole percent of 1,3,5-tris (2-hydroxypropyl)hexahydro-s-triazine. In certain embodiments, the reaction product comprises from 6 to 30 mole percent of 1,3,5-tris(2-hydroxypropyl)hexahydro-s-triazine. In certain embodiments, the reaction product comprises from 7 to 30 mole percent of 1,3,5-tris (2-hydroxypropyl)hexahydro-s-triazine. In certain embodiments, the reaction product comprises from 8 to 30 mole percent of 1,3,5-tris(2-hydroxypropyl)hexahydro-s-triazine. In certain embodiments, the reaction product comprises from 9 to 30 mole percent of 1,3,5-tris (2-hydroxypropyl)hexahydro-s-triazine. In certain embodiments, the reaction product comprises from 10 to 30 mole percent of 1,3,5-tris(2-hydroxypropyl)hexahydro-s-triazine. In certain embodiments, the reaction product comprises from 12 to 30 mole percent of 1,3,5-tris (2-hydroxypropyl)hexahydro-s-triazine. In certain embodiments, the reaction product comprises from 14 to 30 mole percent of 1,3,5-tris(2-hydroxypropyl)hexahydro-s-triazine. In certain embodiments, the reaction product comprises from 16 to 30 mole percent of 1,3,5-tris (2-hydroxypropyl)hexahydro-s-triazine. In certain embodiments, the reaction product comprises from 18 to 30 mole percent of 1,3,5-tris(2-hydroxypropyl)hexahydro-s-triazine. In certain embodiments, the reaction product comprises from 20 to 30 mole percent of 1,3,5-tris (2-hydroxypropyl)hexahydro-s-triazine. In certain embodiments, the reaction product comprises from 22 to 30 mole percent of 1,3,5-tris(2-hydroxypropyl)hexahydro-s-triazine. In certain embodiments, the reaction product comprises from 24 to 30 mole percent of 1,3,5-tris (2-hydroxypropyl)hexahydro-s-triazine. In certain embodiments, the reaction product comprises from 26 to 30 mole percent of 1,3,5-tris(2-hydroxypropyl)hexahydro-s-triazine. In certain embodiments, the reaction product comprises from 28 to 30 mole percent of 1,3,5-tris (2-hydroxypropyl)hexahydro-s-triazine. In certain embodiments, the reaction product comprises from greater than 0 to 28 mole percent of 1,3,5-tris(2-hydroxypropyl)hexahydro-s-triazine. In certain embodiments, the reaction product comprises from 1 to 28 mole percent of 1,3,5-tris (2-hydroxypropyl)hexahydro-s-triazine. In certain embodiments, the reaction product comprises from 2 to 28 mole percent of 1,3,5-tris(2-hydroxypropyl)hexahydro-s-triazine. In certain embodiments, the reaction product comprises from 3 to 28 mole percent of 1,3,5-tris (2-hydroxypropyl)hexahydro-s-triazine. In certain embodiments, the reaction product comprises from 4 to 28 mole percent of 1,3,5-tris(2-hydroxypropyl)hexahydro-s-triazine. In certain embodiments, the reaction product comprises from 5 to 28 mole percent of 1,3,5-tris (2-hydroxypropyl)hexahydro-s-triazine. In certain embodiments, the reaction product comprises from 6 to 28 mole percent of 1,3,5-tris(2-hydroxypropyl)hexahydro-s-triazine. In certain embodiments, the reaction product comprises from 7 to 28 mole percent of 1,3,5-tris (2-hydroxypropyl)hexahydro-s-triazine. In certain embodiments, the reaction product comprises from 8 to 28 mole percent of 1,3,5-tris(2-hydroxypropyl)hexahydro-s-triazine. In certain embodiments, the reaction product comprises from 9 to 28 mole percent of 1,3,5-tris (2-hydroxypropyl)hexahydro-s-triazine. In certain embodiments, the reaction product comprises from 10 to 28 mole percent of 1,3,5-tris(2-hydroxypropyl)hexahydro-s-triazine. In certain embodiments, the reaction product comprises from 12 to 28 mole percent of 1,3,5-tris (2-hydroxypropyl)hexahydro-s-triazine. In certain embodiments, the reaction product comprises from 14 to 28 mole percent of 1,3,5-tris(2-hydroxypropyl)hexahydro-s-triazine. In certain embodiments, the reaction product comprises from 16 to 28 mole percent of 1,3,5-tris (2-hydroxypropyl)hexahydro-s-triazine. In certain embodiments, the reaction product comprises from 18 to 28 mole percent of 1,3,5-tris(2-hydroxypropyl)hexahydro-s-triazine. In certain embodiments, the reaction product comprises from 20 to 28 mole percent of 1,3,5-tris (2-hydroxypropyl)hexahydro-s-triazine. In certain embodiments, the reaction product comprises from 22 to 28 mole percent of 1,3,5-tris(2-hydroxypropyl)hexahydro-s-triazine. In certain embodiments, the reaction product comprises from 24 to 28 mole percent of 1,3,5-tris (2-hydroxypropyl)hexahydro-s-triazine. In certain embodiments, the reaction product comprises from 26 to 28 mole percent of 1,3,5-tris(2-hydroxypropyl)hexahydro-s-triazine. In certain embodiments, the reaction product comprises from greater than 0 to 26 mole percent of 1,3,5-tris(2-hydroxypropyl)hexahydro-s-triazine. In certain embodiments, the reaction product comprises from 1 to 26 mole percent of 1,3,5-tris(2-hydroxypropyl)hexahydro-s-triazine. In certain embodiments, the reaction product comprises from 2 to 26 mole percent of 1,3,5-tris (2-hydroxypropyl)hexahydro-s-triazine. In certain embodiments, the reaction product comprises from 3 to 26 mole percent of 1,3,5-tris(2-hydroxypropyl)hexahydro-s-triazine. In certain embodiments, the reaction product comprises from 4 to 26 mole percent of 1,3,5-tris (2-hydroxypropyl)hexahydro-s-triazine. In certain embodiments, the reaction product comprises from 5 to 26 mole percent of 1,3,5-tris(2-hydroxypropyl)hexahydro-s-triazine. In certain embodiments, the reaction product comprises from 6 to 26 mole percent of 1,3,5-tris (2-hydroxypropyl)hexahydro-s-triazine. In certain embodiments, the reaction product comprises from 7 to 26 mole percent of 1,3,5-tris(2-hydroxypropyl)hexahydro-s-triazine. In certain embodiments, the reaction product comprises from 8 to 26 mole percent of 1,3,5-tris (2-hydroxypropyl)hexahydro-s-triazine. In certain embodiments, the reaction product comprises from 9 to 26 mole percent of 1,3,5-tris(2-hydroxypropyl)hexahydro-s-triazine. In certain embodiments, the reaction product comprises from 10 to 26 mole percent of 1,3,5-tris (2-hydroxypropyl)hexahydro-s-triazine. In certain embodiments, the reaction product comprises from 12 to 26 mole percent of 1,3,5-tris(2-hydroxypropyl)hexahydro-s-triazine. In certain embodiments, the reaction product comprises from 14 to 26 mole percent of 1,3,5-tris (2-hydroxypropyl)hexahydro-s-triazine. In certain embodiments, the reaction product comprises from 16 to 26 mole percent of 1,3,5-tris(2-hydroxypropyl)hexahydro-s-triazine. In certain embodiments, the reaction product comprises from 18 to 26 mole percent of 1,3,5-tris (2-hydroxypropyl)hexahydro-s-triazine. In certain embodiments, the reaction product comprises from 20 to 26 mole percent of 1,3,5-tris(2-hydroxypropyl)hexahydro-s-triazine. In certain embodiments, the reaction product comprises from 22 to 26 mole percent of 1,3,5-tris (2-hydroxypropyl)hexahydro-s-triazine. In certain embodiments, the reaction product comprises from 24 to 26 mole percent of 1,3,5-tris(2-hydroxypropyl)hexahydro-s-triazine. In certain embodiments, the reaction product comprises from greater than 0 to 24 mole percent of 1,3,5-tris(2-hydroxypropyl)hexahydro-s-triazine. In certain embodiments, the reaction product comprises from 1 to 24 mole percent of 1,3,5-tris(2-hydroxypropyl)hexahydro-s-triazine. In certain embodiments, the reaction product comprises from 2 to 24 mole percent of 1,3,5-tris (2-hydroxypropyl)hexahydro-s-triazine. In certain embodiments, the reaction product comprises from 3 to 24 mole percent of 1,3,5-tris(2-hydroxypropyl)hexahydro-s-triazine. In certain embodiments, the reaction product comprises from 4 to 24 mole percent of 1,3,5-tris (2-hydroxypropyl)hexahydro-s-triazine. In certain embodiments, the reaction product comprises from 5 to 24 mole percent of 1,3,5-tris(2-hydroxypropyl)hexahydro-s-triazine. In certain embodiments, the reaction product comprises from 6 to 24 mole percent of 1,3,5-tris (2-hydroxypropyl)hexahydro-s-triazine. In certain embodiments, the reaction product comprises from 7 to 24 mole percent of 1,3,5-tris(2-hydroxypropyl)hexahydro-s-triazine. In certain embodiments, the reaction product comprises from 8 to 24 mole percent of 1,3,5-tris (2-hydroxypropyl)hexahydro-s-triazine. In certain embodiments, the reaction product comprises from 9 to 24 mole percent of 1,3,5-tris(2-hydroxypropyl)hexahydro-s-triazine. In certain embodiments, the reaction product comprises from 10 to 24 mole percent of 1,3,5-tris (2-hydroxypropyl)hexahydro-s-triazine. In certain embodiments, the reaction product comprises from 12 to 24 mole percent of 1,3,5-tris(2-hydroxypropyl)hexahydro-s-triazine. In certain embodiments, the reaction product comprises from 14 to 24 mole percent of 1,3,5-tris (2-hydroxypropyl)hexahydro-s-triazine. In certain embodiments, the reaction product comprises from 16 to 24 mole percent of 1,3,5-tris(2-hydroxypropyl)hexahydro-s-triazine. In certain embodiments, the reaction product comprises from 18 to 24 mole percent of 1,3,5-tris (2-hydroxypropyl)hexahydro-s-triazine. In certain embodiments, the reaction product comprises from 20 to 24 mole percent of 1,3,5-tris(2-hydroxypropyl)hexahydro-s-triazine. In certain embodiments, the reaction product comprises from 22 to 24 mole percent of 1,3,5-tris (2-hydroxypropyl)hexahydro-s-triazine. In certain embodiments, the reaction product comprises from greater than 0 to 22 mole percent of 1,3,5-tris(2-hydroxypropyl)hexahydro-s-triazine. In certain embodiments, the reaction product comprises from 1 to 22 mole percent of 1,3,5-tris (2-hydroxypropyl)hexahydro-s-triazine. In certain embodiments, the reaction product comprises from 2 to 22 mole percent of 1,3,5-tris(2-hydroxypropyl)hexahydro-s-triazine. In certain embodiments, the reaction product comprises from 3 to 22 mole percent of 1,3,5-tris (2-hydroxypropyl)hexahydro-s-triazine. In certain embodiments, the reaction product comprises from 4 to 22 mole percent of 1,3,5-tris(2-hydroxypropyl)hexahydro-s-triazine. In certain embodiments, the reaction product comprises from 5 to 22 mole percent of 1,3,5-tris (2-hydroxypropyl)hexahydro-s-triazine. In certain embodiments, the reaction product comprises from 6 to 22 mole percent of 1,3,5-tris(2-hydroxypropyl)hexahydro-s-triazine. In certain embodiments, the reaction product comprises from 7 to 22 mole percent of 1,3,5-tris (2-hydroxypropyl)hexahydro-s-triazine. In certain embodiments, the reaction product comprises from 8 to 22 mole percent of 1,3,5-tris(2-hydroxypropyl)hexahydro-s-triazine. In certain embodiments, the reaction product comprises from 9 to 22 mole percent of 1,3,5-tris (2-hydroxypropyl)hexahydro-s-triazine. In certain embodiments, the reaction product comprises from 10 to 22 mole percent of 1,3,5-tris(2-hydroxypropyl)hexahydro-s-triazine. In certain embodiments, the reaction product comprises from 12 to 22 mole percent of 1,3,5-tris (2-hydroxypropyl)hexahydro-s-triazine. In certain embodiments, the reaction product comprises from 14 to 22 mole percent of 1,3,5-tris(2-hydroxypropyl)hexahydro-s-triazine. In certain embodiments, the reaction product comprises from 16 to 22 mole percent of 1,3,5-tris (2-hydroxypropyl)hexahydro-s-triazine. In certain embodiments, the reaction product comprises from 18 to 22 mole percent of 1,3,5-tris(2-hydroxypropyl)hexahydro-s-triazine. In certain embodiments, the reaction product comprises from 20 to 22 mole percent of 1,3,5-tris (2-hydroxypropyl)hexahydro-s-triazine. In certain embodiments, the reaction product comprises from greater than 0 to 20 mole percent of 1,3,5-tris(2-hydroxypropyl)hexahydro-s-triazine. In certain embodiments, the reaction product comprises from 1 to 20 mole percent of 1,3,5-tris (2-hydroxypropyl)hexahydro-s-triazine. In certain embodiments, the reaction product comprises from 2 to 20 mole percent of 1,3,5-tris(2-hydroxypropyl)hexahydro-s-triazine. In certain embodiments, the reaction product comprises from 3 to 20 mole percent of 1,3,5-tris (2-hydroxypropyl)hexahydro-s-triazine. In certain embodiments, the reaction product comprises from 4 to 20 mole percent of 1,3,5-tris(2-hydroxypropyl)hexahydro-s-triazine. In certain embodiments, the reaction product comprises from 5 to 20 mole percent of 1,3,5-tris (2-hydroxypropyl)hexahydro-s-triazine. In certain embodiments, the reaction product comprises from 6 to 20 mole percent of 1,3,5-tris(2-hydroxypropyl)hexahydro-s-triazine. In certain embodiments, the reaction product comprises from 7 to 20 mole percent of 1,3,5-tris (2-hydroxypropyl)hexahydro-s-triazine. In certain embodiments, the reaction product comprises from 8 to 20 mole percent of 1,3,5-tris(2-hydroxypropyl)hexahydro-s-triazine. In certain embodiments, the reaction product comprises from 9 to 20 mole percent of 1,3,5-tris (2-hydroxypropyl)hexahydro-s-triazine. In certain embodiments, the reaction product comprises from 10 to 20 mole percent of 1,3,5-tris(2-hydroxypropyl)hexahydro-s-triazine. In certain embodiments, the reaction product comprises from 12 to 20 mole percent of 1,3,5-tris (2-hydroxypropyl)hexahydro-s-triazine. In certain embodiments, the reaction product comprises from 14 to 20 mole percent of 1,3,5-tris(2-hydroxypropyl)hexahydro-s-triazine. In certain embodiments, the reaction product comprises from 16 to 20 mole percent of 1,3,5-tris (2-hydroxypropyl)hexahydro-s-triazine. In certain embodiments, the reaction product comprises from 18 to 20 mole percent of 1,3,5-tris(2-hydroxypropyl)hexahydro-s-triazine. In certain embodiments, the reaction product comprises from greater than 0 to 18 mole percent of 1,3,5-tris(2-hydroxypropyl)hexahydro-s-triazine. In certain embodiments, the reaction product comprises from 1 to 18 mole percent of 1,3,5-tris(2-hydroxypropyl)hexahydro-s-triazine. In certain embodiments, the reaction product comprises from 2 to 18 mole percent of 1,3,5-tris (2-hydroxypropyl)hexahydro-s-triazine. In certain embodiments, the reaction product comprises from 3 to 18 mole percent of 1,3,5-tris(2-hydroxypropyl)hexahydro-s-triazine. In certain embodiments, the reaction product comprises from 4 to 18 mole percent of 1,3,5-tris (2-hydroxypropyl)hexahydro-s-triazine. In certain embodiments, the reaction product comprises from 5 to 18 mole percent of 1,3,5-tris(2-hydroxypropyl)hexahydro-s-triazine. In certain embodiments, the reaction product comprises from 6 to 18 mole percent of 1,3,5-tris (2-hydroxypropyl)hexahydro-s-triazine. In certain embodiments, the reaction product comprises from 7 to 18 mole percent of 1,3,5-tris(2-hydroxypropyl)hexahydro-s-triazine. In certain embodiments, the reaction product comprises from 8 to 18 mole percent of 1,3,5-tris (2-hydroxypropyl)hexahydro-s-triazine. In certain embodiments, the reaction product comprises from 9 to 18 mole percent of 1,3,5-tris(2-hydroxypropyl)hexahydro-s-triazine. In certain embodiments, the reaction product comprises from 10 to 18 mole percent of 1,3,5-tris (2-hydroxypropyl)hexahydro-s-triazine. In certain embodiments, the reaction product comprises from 12 to 18 mole percent of 1,3,5-tris(2-hydroxypropyl)hexahydro-s-triazine. In certain embodiments, the reaction product comprises from 14 to 18 mole percent of 1,3,5-tris (2-hydroxypropyl)hexahydro-s-triazine. In certain embodiments, the reaction product comprises from 16 to 18 mole percent of 1,3,5-tris(2-hydroxypropyl)hexahydro-s-triazine. In certain embodiments, the reaction product comprises from greater than 0 to 16 mole percent of 1,3,5-tris(2-hydroxypropyl)hexahydro-s-triazine. In certain embodiments, the reaction product comprises from 1 to 16 mole percent of 1,3,5-tris(2-hydroxypropyl)hexahydro-s-triazine. In certain embodiments, the reaction product comprises from 2 to 16 mole percent of 1,3,5-tris (2-hydroxypropyl)hexahydro-s-triazine. In certain embodiments, the reaction product comprises from 3 to 16 mole percent of 1,3,5-tris(2-hydroxypropyl)hexahydro-s-triazine. In certain embodiments, the reaction product comprises from 4 to 16 mole percent of 1,3,5-tris (2-hydroxypropyl)hexahydro-s-triazine. In certain embodiments, the reaction product comprises from 5 to 16 mole percent of 1,3,5-tris(2-hydroxypropyl)hexahydro-s-triazine. In certain embodiments, the reaction product comprises from 6 to 16 mole percent of 1,3,5-tris (2-hydroxypropyl)hexahydro-s-triazine. In certain embodiments, the reaction product comprises from 7 to 16 mole percent of 1,3,5-tris(2-hydroxypropyl)hexahydro-s-triazine. In certain embodiments, the reaction product comprises from 8 to 16 mole percent of 1,3,5-tris (2-hydroxypropyl)hexahydro-s-triazine. In certain embodiments, the reaction product comprises from 9 to 16 mole percent of 1,3,5-tris(2-hydroxypropyl)hexahydro-s-triazine. In certain embodiments, the reaction product comprises from 10 to 16 mole percent of 1,3,5-tris (2-hydroxypropyl)hexahydro-s-triazine. In certain embodiments, the reaction product comprises from 12 to 16 mole percent of 1,3,5-tris(2-hydroxypropyl)hexahydro-s-triazine. In certain embodiments, the reaction product comprises from 14 to 16 mole percent of 1,3,5-tris (2-hydroxypropyl)hexahydro-s-triazine. In certain embodiments, the reaction product comprises from greater than 0 to 14 mole percent of 1,3,5-tris(2-hydroxypropyl)hexahydro-s-triazine. In certain embodiments, the reaction product comprises from 1 to 14 mole percent of 1,3,5-tris (2-hydroxypropyl)hexahydro-s-triazine. In certain embodiments, the reaction product comprises from 2 to 14 mole percent of 1,3,5-tris(2-hydroxypropyl)hexahydro-s-triazine. In certain embodiments, the reaction product comprises from 3 to 14 mole percent of 1,3,5-tris (2-hydroxypropyl)hexahydro-s-triazine. In certain embodiments, the reaction product comprises from 4 to 14 mole percent of 1,3,5-tris(2-hydroxypropyl)hexahydro-s-triazine. In certain embodiments, the reaction product comprises from 5 to 14 mole percent of 1,3,5-tris (2-hydroxypropyl)hexahydro-s-triazine. In certain embodiments, the reaction product comprises from 6 to 14 mole percent of 1,3,5-tris(2-hydroxypropyl)hexahydro-s-triazine. In certain embodiments, the reaction product comprises from 7 to 14 mole percent of 1,3,5-tris (2-hydroxypropyl)hexahydro-s-triazine. In certain embodiments, the reaction product comprises from 8 to 14 mole percent of 1,3,5-tris(2-hydroxypropyl)hexahydro-s-triazine. In certain embodiments, the reaction product comprises from 9 to 14 mole percent of 1,3,5-tris (2-hydroxypropyl)hexahydro-s-triazine. In certain embodiments, the reaction product comprises from 10 to 14 mole percent of 1,3,5-tris(2-hydroxypropyl)hexahydro-s-triazine. In certain embodiments, the reaction product comprises from 12 to 14 mole percent of 1,3,5-tris (2-hydroxypropyl)hexahydro-s-triazine. In certain embodiments, the reaction product comprises from greater than 0 to 12 mole percent of 1,3,5-tris(2-hydroxypropyl)hexahydro-s-triazine. In certain embodiments, the reaction product comprises from 1 to 12 mole percent of 1,3,5-tris (2-hydroxypropyl)hexahydro-s-triazine. In certain embodiments, the reaction product comprises from 2 to 12 mole percent of 1,3,5-tris(2-hydroxypropyl)hexahydro-s-triazine. In certain embodiments, the reaction product comprises from 3 to 12 mole percent of 1,3,5-tris (2-hydroxypropyl)hexahydro-s-triazine. In certain embodiments, the reaction product comprises from 4 to 12 mole percent of 1,3,5-tris(2-hydroxypropyl)hexahydro-s-triazine. In certain embodiments, the reaction product comprises from 5 to 12 mole percent of 1,3,5-tris (2-hydroxypropyl)hexahydro-s-triazine. In certain embodiments, the reaction product comprises from 6 to 12 mole percent of 1,3,5-tris(2-hydroxypropyl)hexahydro-s-triazine. In certain embodiments, the reaction product comprises from 7 to 12 mole percent of 1,3,5-tris (2-hydroxypropyl)hexahydro-s-triazine. In certain embodiments, the reaction product comprises from 8 to 12 mole percent of 1,3,5-tris(2-hydroxypropyl)hexahydro-s-triazine. In certain embodiments, the reaction product comprises from 9 to 12 mole percent of 1,3,5-tris (2-hydroxypropyl)hexahydro-s-triazine. In certain embodiments, the reaction product comprises from 10 to 12 mole percent of 1,3,5-tris(2-hydroxypropyl)hexahydro-s-triazine. In certain embodiments, the reaction product comprises from greater than 0 to 10 mole percent of 1,3,5-tris(2-hydroxypropyl)hexahydro-s-triazine. In certain embodiments, the reaction product comprises from 1 to 10 mole percent of 1,3,5-tris(2-hydroxypropyl)hexahydro-s-triazine. In certain embodiments, the reaction product comprises from 2 to 10 mole percent of 1,3,5-tris (2-hydroxypropyl)hexahydro-s-triazine. In certain embodiments, the reaction product comprises from 3 to 10 mole percent of 1,3,5-tris(2-hydroxypropyl)hexahydro-s-triazine. In certain embodiments, the reaction product comprises from 4 to 10 mole percent of 1,3,5-tris (2-hydroxypropyl)hexahydro-s-triazine. In certain embodiments, the reaction product comprises from 5 to 10 mole percent of 1,3,5-tris(2-hydroxypropyl)hexahydro-s-triazine. In certain embodiments, the reaction product comprises from 6 to 10 mole percent of 1,3,5-tris (2-hydroxypropyl)hexahydro-s-triazine. In certain embodiments, the reaction product comprises from 7 to 10 mole percent of 1,3,5-tris(2-hydroxypropyl)hexahydro-s-triazine. In certain embodiments, the reaction product comprises from 8 to 10 mole percent of 1,3,5-tris (2-hydroxypropyl)hexahydro-s-triazine. In certain embodiments, the reaction product comprises from 9 to 10 mole percent of 1,3,5-tris(2-hydroxypropyl)hexahydro-s-triazine. In certain embodiments, the reaction product comprises from greater than 0 to 9 mole percent of 1,3,5-tris(2-hydroxypropyl)hexahydro-s-triazine. In certain embodiments, the reaction product comprises from 1 to 9 mole percent of 1,3,5-tris(2-hydroxypropyl)hexahydro-s-triazine. In certain embodiments, the reaction product comprises from 2 to 9 mole percent of 1,3,5-tris (2-hydroxypropyl)hexahydro-s-triazine. In certain embodiments, the reaction product comprises from 3 to 9 mole percent of 1,3,5-tris(2-hydroxypropyl)hexahydro-s-triazine. In certain embodiments, the reaction product comprises from 4 to 9 mole percent of 1,3,5-tris (2-hydroxypropyl)hexahydro-s-triazine. In certain embodiments, the reaction product comprises from 5 to 9 mole percent of 1,3,5-tris(2-hydroxypropyl)hexahydro-s-triazine. In certain embodiments, the reaction product comprises from 6 to 9 mole percent of 1,3,5-tris (2-hydroxypropyl)hexahydro-s-triazine. In certain embodiments, the reaction product comprises from 7 to 9 mole percent of 1,3,5-tris(2-hydroxypropyl)hexahydro-s-triazine. In certain embodiments, the reaction product comprises from 8 to 9 mole percent of 1,3,5-tris (2-hydroxypropyl)hexahydro-s-triazine. In certain embodiments, the reaction product comprises from greater than 0 to 8 mole percent of 1,3,5-tris(2-hydroxypropyl)hexahydro-s-triazine. In certain embodiments, the reaction product comprises from 1 to 8 mole percent of 1,3,5-tris (2-hydroxypropyl)hexahydro-s-triazine. In certain embodiments, the reaction product comprises from 2 to 8 mole percent of 1,3,5-tris(2-hydroxypropyl)hexahydro-s-triazine. In certain embodiments, the reaction product comprises from 3 to 8 mole percent of 1,3,5-tris (2-hydroxypropyl)hexahydro-s-triazine. In certain embodiments, the reaction product comprises from 4 to 8 mole percent of 1,3,5-tris(2-hydroxypropyl)hexahydro-s-triazine. In certain embodiments, the reaction product comprises from 5 to 8 mole percent of 1,3,5-tris (2-hydroxypropyl)hexahydro-s-triazine. In certain embodiments, the reaction product comprises from 6 to 8 mole percent of 1,3,5-tris(2-hydroxypropyl)hexahydro-s-triazine. In certain embodiments, the reaction product comprises from 7 to 8 mole percent of 1,3,5-tris (2-hydroxypropyl)hexahydro-s-triazine. In certain embodiments, the reaction product comprises from greater than 0 to 7 mole percent of 1,3,5-tris(2-hydroxypropyl)hexahydro-s-triazine. In certain embodiments, the reaction product comprises from 1 to 7 mole percent of 1,3,5-tris (2-hydroxypropyl)hexahydro-s-triazine. In certain embodiments, the reaction product comprises from 2 to 7 mole percent of 1,3,5-tris(2-hydroxypropyl)hexahydro-s-triazine. In certain embodiments, the reaction product comprises from 3 to 7 mole percent of 1,3,5-tris (2-hydroxypropyl)hexahydro-s-triazine. In certain embodiments, the reaction product comprises from 4 to 7 mole percent of 1,3,5-tris(2-hydroxypropyl)hexahydro-s-triazine. In certain embodiments, the reaction product comprises from 5 to 7 mole percent of 1,3,5-tris (2-hydroxypropyl)hexahydro-s-triazine. In certain embodiments, the reaction product comprises from 6 to 7 mole percent of 1,3,5-tris(2-hydroxypropyl)hexahydro-s-triazine. In certain embodiments, the reaction product comprises from greater than 0 to 6 mole percent of 1,3,5-tris(2-hydroxypropyl)hexahydro-s-triazine. In certain embodiments, the reaction product comprises from 1 to 6 mole percent of 1,3,5-tris(2-hydroxypropyl)hexahydro-s-triazine. In certain embodiments, the reaction product comprises from 2 to 6 mole percent of 1,3,5-tris (2-hydroxypropyl)hexahydro-s-triazine. In certain embodiments, the reaction product comprises from 3 to 6 mole percent of 1,3,5-tris(2-hydroxypropyl)hexahydro-s-triazine. In certain embodiments, the reaction product comprises from 4 to 6 mole percent of 1,3,5-tris (2-hydroxypropyl)hexahydro-s-triazine. In certain embodiments, the reaction product comprises from 5 to 6 mole percent of 1,3,5-tris(2-hydroxypropyl)hexahydro-s-triazine. In certain embodiments, the reaction product comprises from greater than 0 to 5 mole percent of 1,3,5-tris(2-hydroxypropyl)hexahydro-s-triazine. In certain embodiments, the reaction product comprises from 1 to 5 mole percent of 1,3,5-tris(2-hydroxypropyl)hexahydro-s-triazine. In certain embodiments, the reaction product comprises from 2 to 5 mole percent of 1,3,5-tris (2-hydroxypropyl)hexahydro-s-triazine. In certain embodiments, the reaction product comprises from 3 to 5 mole percent of 1,3,5-tris(2-hydroxypropyl)hexahydro-s-triazine. In certain embodiments, the reaction product comprises from 4 to 5 mole percent of 1,3,5-tris (2-hydroxypropyl)hexahydro-s-triazine. In certain embodiments, the reaction product comprises from greater than 0 to 4 mole percent of 1,3,5-tris(2-hydroxypropyl)hexahydro-s-triazine. In certain embodiments, the reaction product comprises from 1 to 4 mole percent of 1,3,5-tris (2-hydroxypropyl)hexahydro-s-triazine. In certain embodiments, the reaction product comprises from 2 to 4 mole percent of 1,3,5-tris(2-hydroxypropyl)hexahydro-s-triazine. In certain embodiments, the reaction product comprises from 3 to 4 mole percent of 1,3,5-tris (2-hydroxypropyl)hexahydro-s-triazine. In certain embodiments, the reaction product comprises from greater than 0 to 3 mole percent of 1,3,5-tris(2-hydroxypropyl)hexahydro-s-triazine. In certain embodiments, the reaction product comprises from 1 to 3 mole percent of 1,3,5-tris (2-hydroxypropyl)hexahydro-s-triazine. In certain embodiments, the reaction product comprises from 2 to 3 mole percent of 1,3,5-tris(2-hydroxypropyl)hexahydro-s-triazine. In certain embodiments, the reaction product comprises from greater than 0 to 2 mole percent of 1,3,5-tris(2-hydroxypropyl)hexahydro-s-triazine. In certain embodiments, the reaction product comprises from 1 to 2 mole percent of 1,3,5-tris(2-hydroxypropyl)hexahydro-s-triazine. In certain embodiments, the reaction product comprises from greater than 0 to 1 mole percent of 1,3,5-tris(2-hydroxypropyl)hexahydro-s-triazine.

In certain embodiments, the reaction product has a water content of 10 percent by weight or less, based on the total weight of the reaction product. Without limitation, there may be a few reasons to limit the amount of water in the reaction product. First, a lower water content may result in a more stable reaction product, meaning that the reaction product is chemically stable and/or shelf-stable for long-term storage. Second, in certain applications, it may be unsafe to have a significant amount of water in the reaction product, such as when the reaction product is added to a substantially water-free refinery stream which is at high temperature.

In certain embodiments, the reaction product has a water content of 9 percent by weight or less. In certain embodiment, the reaction product has a water content of 8 percent by weight or less. In certain embodiments, the reaction product has a water content of 7 percent by weight or less. In certain embodiments, the reaction product has a water content of 6 percent by weight or less. In certain embodiments, the reaction product has a water content of 5 percent by weight or less. In certain embodiments, the reaction product has a water content of 4 percent by weight or less. In certain embodiments, the reaction product has a water content of 3 percent by weight or less. In certain embodiments, the reaction product has a water content of 2 percent by weight or less. In certain embodiments, the reaction product has a water content of 1 percent by weight or less. In certain embodiments, the reaction product has a water content of greater than 0 to 10 percent by weight. In certain embodiments, the reaction product has a water content of greater than 0 to 9 percent by weight. In certain embodiments, the reaction product has a water content of greater than 0 to 8 percent by weight. In certain embodiments, the reaction product has a water content of greater than 0 to 7 percent by weight. In certain embodiments, the reaction product has a water content of greater than 0 to 6 percent by weight. In certain embodiments, the reaction product has a water content of greater than 0 to 5 percent by weight. In certain embodiments, the reaction product has a water content of greater than 0 to 4 percent by weight. In certain embodiments, the reaction product has a water content of greater than 0 to 3 percent by weight. In certain embodiments, the reaction product has a water content of greater than 0 to 2 percent by weight. In certain embodiments, the reaction product has a water content of greater than 0 to 1 percent by weight. In certain embodiments, the reaction product has a water content of 1 to 10 percent by weight. In certain embodiments, the reaction product has a water content of 1 to 9 percent by weight. In certain embodiments, the reaction product has a water content of 1 to 8 percent by weight. In certain embodiments, the reaction product has a water content of 1 to 7 percent by weight. In certain embodiments, the reaction product has a water content of 1 to 6 percent by weight. In certain embodiments, the reaction product has a water content of 1 to 5 percent by weight. In certain embodiments, the reaction product has a water content of 1 to 4 percent by weight. In certain embodiments, the reaction product has a water content of 1 to 3 percent by weight. In certain embodiments, the reaction product has a water content of 1 to 2 percent by weight. In certain embodiments, the reaction product has a water content of 2 to 10 percent by weight. In certain embodiments, the reaction product has a water content of 2 to 9 percent by weight. In certain embodiments, the reaction product has a water content of 2 to 8 percent by weight. In certain embodiments, the reaction product has a water content of 2 to 7 percent by weight. In certain embodiments, the reaction product has a water content of 2 to 6 percent by weight. In certain embodiments, the reaction product has a water content of 2 to 5 percent by weight. In certain embodiments, the reaction product has a water content of 2 to 4 percent by weight. In certain embodiments, the reaction product has a water content of 2 to 3 percent by weight. In certain embodiments, the reaction product has a water content of 3 to 10 percent by weight. In certain embodiments, the reaction product has a water content of 3 to 9 percent by weight. In certain embodiments, the reaction product has a water content of 3 to 8 percent by weight. In certain embodiments, the reaction product has a water content of 3 to 7 percent by weight. In certain embodiments, the reaction product has a water content of 3 to 6 percent by weight. In certain embodiments, the reaction product has a water content of 3 to 5 percent by weight. In certain embodiments, the reaction product has a water content of 3 to 4 percent by weight. In certain embodiments, the reaction product has a water content of 4 to 10 percent by weight. In certain embodiments, the reaction product has a water content of 4 to 9 percent by weight. In certain embodiments, the reaction product has a water content of 4 to 8 percent by weight. In certain embodiments, the reaction product has a water content of 4 to 7 percent by weight. In certain embodiments, the reaction product has a water content of 4 to 6 percent by weight. In certain embodiments, the reaction product has a water content of 4 to 5 percent by weight. In certain embodiments, the reaction product has a water content of 5 to 10 percent by weight. In certain embodiments, the reaction product has a water content of 5 to 9 percent by weight. In certain embodiments, the reaction product has a water content of 5 to 8 percent by weight. In certain embodiments, the reaction product has a water content of 5 to 7 percent by weight. In certain embodiments, the reaction product has a water content of 5 to 6 percent by weight. In certain embodiments, the reaction product has a water content of 6 to 10 percent by weight. In certain embodiments, the reaction product has a water content of 6 to 9 percent by weight. In certain embodiments, the reaction product has a water content of 6 to 8 percent by weight. In certain embodiments, the reaction product has a water content of 6 to 7 percent by weight. In certain embodiments, the reaction product has a water content of 7 to 10 percent by weight. In certain embodiments, the reaction product has a water content of 7 to 9 percent by weight. In certain embodiments, the reaction product has a water content of 7 to 8 percent by weight. In certain embodiments, the reaction product has a water content of 8 to 10 percent by weight. In certain embodiments, the reaction product has a water content of 8 to 9 percent by weight. In certain embodiments, the reaction product has a water content of 9 to 10 percent by weight.

Also provided are compositions prepared according to the processes described herein.

Further provided are compositions comprising a reaction product (as described herein), wherein the reaction product is prepared by adding 1-amino-2-propanol into a liquid form of formaldehyde to form an intermediate reaction mixture, wherein the molar ratio of formaldehyde to 1-amino-2-propanol is from 1.4:1 to less than 2:1, and subsequently subjecting the intermediate reaction mixture to reactive distillation to form the reaction product, wherein the predominant compound present in the reaction product is 3,3′-methylenebis[5-methyloxazolidine].

In certain embodiments, the reaction product(s) described herein may be used for removing sulfur-containing compounds from fluids, such as process streams. The fluid may be a liquid or a gas. The process stream may be a liquid process stream or a gaseous process stream. In certain embodiments, the fluid and/or the process stream may comprise water and/or hydrocarbon. In certain embodiments, the sulfur-containing compound may comprise at least one of hydrogen sulfide, inorganic sulfides, organic sulfides, mercaptans, or mercaptides.

In certain embodiments, provided is use of the reaction product(s) described herein for removing sulfur-containing compounds from fluids, such as process streams. The fluid may be a liquid or a gas. The process stream may be a liquid process stream or a gaseous process stream. In certain embodiments, the fluid and/or the process stream may comprise water and/or hydrocarbon. In certain embodiments, the sulfur-containing compound may comprise at least one of hydrogen sulfide, inorganic sulfides, organic sulfides, mercaptans, or mercaptides.

In certain embodiments, provided are methods of removing one or more sulfur-containing compounds from a fluid, such as a process stream, the methods comprising bringing the process stream into contact with the reaction product(s) described herein. The fluid may be a liquid or a gas. The process stream may be a liquid process stream or a gaseous process stream. In certain embodiments, the fluid and/or the process stream may comprise water and/or hydrocarbon. In certain embodiments, the sulfur-containing compound may comprise at least one of hydrogen sulfide, inorganic sulfides, organic sulfides, mercaptans, or mercaptides.

The subject matter disclosed herein may be better understood with reference to the following examples, which are set forth merely to further illustrate the subject matter disclosed herein. The illustrative examples should not be construed as limiting the subject matter in any manner.

Example 1:1-Amino-2-propanol was reacted with a formalin solution (37 wt % formaldehyde and 12 wt % methanol in water) in a continuous reactor having a baffled internal path to ensure intimate mixing of reactants. The molar ratio of contained formaldehyde to 1-amino-2-propanol was 1.60:1. The 1-amino-2-propanol was injected in equal thirds at three equally separated points along the path of the continuous reactor. The continuous reactor had an external cooling jacket for temperature control. The internal temperature of the continuous reactor was maintained at less than 80° C. The intermediate reaction mixture exiting the continuous reactor was fed into the middle of a distillation column having a packed bed equivalent to twelve theoretical stages and equipped with a reboiler at the bottom. The temperature in the reboiler was maintained at 120° C., the pressure in the distillation column was maintained at 73 torr, and the reflux ratio in the column was 0.5. The product from the outlet of the reboiler was a clear, colorless liquid that gave a clear solution with no separation when mixed 1:1 by weight with isooctane at room temperature. The composition of the product was determined by integration of the 13C NMR spectrum in d6-dimethylsulfoxide solution. The region of the 13C spectrum from 17.5 ppm to 22.0 ppm (relative to a hexamethyldisiloxane standard at 0) is the region where the methyl groups from incorporation of 1-amino-2-propanol appear. The largest peak in this region, at 20.0 ppm, is 3,3′-methylenebis[5-methyloxazolidine], representing 62.6% of the total peak area integration of this region. Similarly, 3-hydroxymethyl-5-methyloxazolidine at 20.8 ppm represents 5.7% of the total methyl peak area, 1,3,5-tris(2-hydroxypropyl)hexahydro-s-triazine at 21.5 ppm represents 1.5% of the total methyl peak area, and 3,3′-(oxybis(methylene))bis(5-methyloxazolidine) at 18.44 ppm represents 3.6% of the total area. The water content of the product was 0.12% as measured by Karl Fisher titration.

Except in the Example, or where otherwise explicitly indicated or required by context, all numerical quantities in this description specifying amounts of materials, reaction conditions, molecular weights, number of carbon atoms, and the like, are to be understood as modified by the word “about”. As used herein, the term “about” means that a value of a given quantity is within ±20% of the stated value. In other embodiments, the value is within ±15% of the stated value. In other embodiments, the value is within ±10% of the stated value. In other embodiments, the value is within +5% of the stated value. In other embodiments, the value is within ±2.5% of the stated value. In other embodiments, the value is within ±1% of the stated value. In other embodiments, the value is within a range of the explicitly-described value which would be understood by those of ordinary skill, based on the disclosures provided herein, to perform substantially similarly to compositions including the literal amounts described herein.

It is to be understood that the upper and lower amount, range, and ratio limits set forth herein may be independently combined, and that any amount within a disclosed range is contemplated to provide a minimum or maximum of a narrower range in alternative embodiments (with the proviso, of course, that the minimum amount of a range must be lower than the maximum amount of the same range). Similarly, the ranges and amounts for each element of the subject matter disclosed herein may be used together with ranges or amounts for any of the other elements.

While certain representative embodiments and details have been shown for the purpose of illustrating the subject matter disclosed herein, it will be apparent to those skilled in this art that various changes and modifications may be made therein without departing from the scope of the subject matter. In this regard, the scope of the invention is to be limited only by the following claims.

1. A process for preparing a reaction product, the process comprising adding 1-amino-2-propanol into a liquid form of formaldehyde to form an intermediate reaction mixture, wherein the molar ratio of formaldehyde to 1-amino-2-propanol is from 1.4:1 to less than 2:1, and subsequently subjecting the intermediate reaction mixture to reactive distillation to form the reaction product, wherein the predominant compound present in the reaction product is 3,3′-methylenebis[5-methyloxazolidine].

2. The process of claim 1, wherein formation of the intermediate reaction mixture is substantially complete instantaneously after adding the 1-amino-2-propanol into the liquid form of formaldehyde.

3. The process of claim 1, wherein the reaction product comprises less than 30 percent by weight of 1,3,5-tris(2-hydroxypropyl)hexahydro-s-triazine, based on the total weight of the reaction product.

4. The process of claim 1, wherein the reaction product has a water content of 10 percent by weight or less, based on the total weight of the reaction product.

5. A composition prepared according to the process of claim 1.

6. A composition comprising a reaction product, wherein the reaction product is prepared by adding 1-amino-2-propanol into a liquid form of formaldehyde to form an intermediate reaction mixture, wherein the molar ratio of formaldehyde to 1-amino-2-propanol is from 1.4:1 to less than 2:1, and subsequently subjecting the intermediate reaction mixture to reactive distillation to form the reaction product, wherein the predominant compound present in the reaction product is 3,3′-methylenebis[5-methyloxazolidine].

7. The composition of claim 6, wherein the reaction product comprises less than 30 percent by weight of 1,3,5-tris(2-hydroxypropyl)hexahydro-s-triazine, based on the total weight of the reaction product.

8. The composition of claim 6, wherein the reaction product has a water content of 10 percent by weight or less, based on the total weight of the reaction product.
